SEQUENCE LISTING (4 sequences)

SEQ ID NO: 1 - DNA, 4696 bp, Homo sapiens
aaaaccaccagggttgttgctggaaagtttttattcctggattaaaggcaaggatcagcctgtattttagcaatttctttttaaggttaatgtcccatgcgccacctacttctggggccctgttccagcccttctttatgtgttgaccacttctaggtccagcacttcccaactctgctgcgcagtggactcaatcccctgggaagtcctttaaaaatgcccaagtcagcccccgcctacccccaaagatgcatggaccagaaatctctgaaaggtggcctgagtattactattttctaaaaggctctctcagaccattttaatgggcacccagtgttgaaaataactgctccagtttgttaaaaaataattggtgtgaatattggcaaaagccctctggcacaaagaaagagaaccagtttcttctagctaatgtttgttagccagaattatctgtggcatagtccatgtgacttaatagacctggtcttccagggcagctgaatgcaaatgtttctcacgtgtagaacgggatgtcagggcttacagagaaagtgggaaactggaatgatgactccatctaattcggccatgctggatgattcacctggattctctcatgtcctgagcattgaaaacataatgaagagtttttaaattgaatgtttaaaagagtgaaaacaactccatccctttttctgtttccttttaccttgtatttatgtaccaccaggtaccttgctcttggcagtgagcgtgaatgaatggcacagctcagcccctgaagcctgtgtgcagagattgagggattgtgatggagtagttcattcatgctcatgttaaggggggtgctaatagcagactagtgctcctgcgattattaatatctaggtctgggacagattgtgatggcttcttttccagttgccacctcagcagaaagggaaatagaaaaccctaacttgtaaagttagacaattagactgtaaagtttgtatatgtgacaacttcagatacaaagacacacacttacccttgacggggcttaagaggagagtgtcaaacataataccaaagtgaaagaagatagctcttcatctacaaattatttttaaacacatttaccaggttaaacaataactaatttttcggaagagaagagtacccaaagtcaaatgccctaagacgaagagatgcttatggcatttttttttaaataaagaaaatgcaaagttagagtggttctgaaggaacctaggatgaataaggtacagacatgattattctaatggtgcagacaggattgagagagaaggggggaggggagagatggagaaaggcatggatggaagatgacgtttggattcagattttggaaaggagagtaaaggaaggaggtaagcagagatttattttttaaattttattaatgtgttttcccctctttttcttgttatttttctcatctgtctgttcatacttggatattttgtccaataaactatcttctaaggactctgaaaatgcactgaatatttttggagggtttactggggtgccagacgccactttaggagttttacatatcctctccatttcatttagttctcttagcacagagaagtgggagaagatagtcccattttacaggtgggatgaagagagagatggaggaatttgccccaggttactcagctagaaggtggtgaagaactcaagccttcggatatcagcgcctggcatttaactaccaatcggtcctgctgggactccggctcctctggcaccatccccgggacctactcagagagtttgcacgtggccggtcgcgttccatcgtctaacaaggtccagcacagcgcaaatccgaagatcgtctaccccggggaaaaagagagtctgtttaattctcctgtggccctccaagtgagttcttttgggttccattgcctagacgaggaaagtgaggctttgcctgctctgcgctcacagggtcggcaagtagtgggaccctaggttcctgcagtattccagagataatcaaagctgcacaggtctcgtcatttttatgcaaaggcgtccggaaggctcgaactctcccttgcacaagcccatctgtctctgtgcgccgcccccgggacacggaagcaggcggcgagcagcgccgagtgggtggagaaccgtcccccgccactcacccctcggccaactctccgcgccttctcagccggcacccacgaggccgacctctctcggcctaaaaaaaaaaaaaaaaaatcccggcctcccctgcaccccgcccgccgcccccagggagctgcattaatattaatctcgctgaataattgaaggccagagatttattcgagcttcggcgggggagggagcgcagctgggccgcgtttaggctgcaccacccgcgtgtttcagccgctcgactccgctggacctgggacccccagacgtgggaggatggggtgggtgtgcctgcctgtgagtttgggggtgagtgtgagctgaagcgggtgctccggggagtgaggagggagcgccaggggctgctccagggaggcggagacggaggggcatcccgggtctccgcgcggtcgcctgcgcttcaccccgcacggggtgacctggggccacgcgggcttcaggggaaacaatagctactccttagatcctgggctcctgccaccggctgcccaagccttcccggacgagcggcggggcctcttttcttatttggctaatttatggcgagaggctgggggaagggatggcagaggagggaccgcgactgaaaatgggggcggggggcggcggttaaaggagttgcccgaggcggcggcgcgggtgatgtcagctctcgacgaaaatagagagggatcgcctgcaaatccccagctccggcggggctaaaccttgcaatccctccctggccggcgccgagccagagcgcagcggcctccaccgcctccccaggcgcgcacacacccgcacacgcgcacgcacgctcaccgtcctctgccaccactctctgctcccgccactcgccgcgcccgcgagccccgcagcaaagcacaggtggcagcggctgcaggggcgcatcgccggcgtgcgccctcctgcagccctgggcgcatcgctctctcggggaagccaccctcggagcccccggagctccccgccaagcgccatccccgcgggcggaggggagcgcgggtcgcgcgccgtggagagccgggacgcggattagcgcccgcaggagcctcctgcgcccgttgaggcgctaaagggcttaccccggaggcgggtggaagggcgggcagaggctcctcttaaataccgctcccggccgcacttcgcgctcaccccggcgtccgctttctccctcgcccacagctgccggatagtgctgaagaggagggggcgttccccagaccatggcatctacggaagggtgaggggatttttatctgtacccgcgggaaagcggggtcacgcgcggggtggtggcgcccctatccgggatgcggatagagaggcggcggcggcgggcctcggaggtggtggcggagccgtagcttggctggggatgggatggtggggaggggattgattttctttcctggagattgctgcttaatcctttgaaaatgcgagaggtggagggttgttttattttgataaaaagggtaaggtgcgctgggggcctgagagtgtgaggaagaaatcctcttgaggttacttttgggatttcaaaacaataggggattgggcatagtgtgagcagacaccggggtagcagcgcctggagcgcggcgccccaggcccgaggcgggcttgcaggtggtgccggctcggaaggaatgagccaagacagggccctggggcggggcaaggaccagcgcgcgcggccttgaacgccaggtttgcagagtcgccatggagatgctgggcccgctccgatcggtccttgtccctggaaggcggaatctccctggctagctctaaggaagggtggaagagatttgggtgcttcccgggaggcgggaaaacgtgtggtttgggacaagggcaggagtcgccagactccagcgggcagggatagcattggcttccctattcagcccgaggatctggagtcgtgtcctgcctcccaagattccagctggcatggggaaagctccctcgcagtgataactaaagacaattgtctttagcaagagacagaaggggctgcagggggcaaaaggattctttgaatactcacacatcaaaggaaaggtccacagagtccttggaccagtatctcccagaaaactttttgggcttcgtagaacctgagtggcaatgaaaagactgggcagctcagccctttggttaattcccaaaattgcagttactcacttgcaagcgatcacaaaatccatgttatgtgaaaagcaaatatcaggggcttctctgggctcaagtggtggtgttggcattttccagtttctcctaagaaattttaccaactccgcaggcttgttttaggggaatggatctctaaacaggctgaagagctggtatccaaagccagatctctagactgcaatctccaatagaaggaaaatatttctagaactgtctctctgtccaggagaaggaattccagcacactggcggccgttactagtggatccgagct

SEQ ID NO: 2 - DNA, 2718 bp, Homo sapiens
ggtaccttgctcttggcagtgagcgtgaatgaatggcacagctcagcccctgaagcctgtgtgcagagattgagggattgtgatggagtagttcattcatgctcatgttaaggggggtgctaatagcagactagtgctcctgcgattattaatatctaggtctgggacagattgtgatggcttcttttccagttgccacctcagcagaaagggaaatagaaaaccctaacttgtaaagttagacaattagactgtaaagtttgtatatgtgacaacttcagatacaaagacacacacttacccttgacggggcttaagaggagagtgtcaaacataataccaaagtgaaagaagatagctcttcatctacaaattatttttaaacacatttaccaggttaaacaataactaatttttcggaagagaagagtacccaaagtcaaatgccctaagacgaagagatgcttatggcatttttttttaaataaagaaaatgcaaagttagagtggttctgaaggaacctaggatgaataaggtacagacatgattattctaatggtgcagacaggattgagagagaaggggggaggggagagatggagaaaggcatggatggaagatgacgtttggattcagattttggaaaggagagtaaaggaaggaggtaagcagagatttattttttaaattttattaatgtgttttcccctctttttcttgttatttttctcatctgtctgttcatacttggatattttgtccaataaactatcttctaaggactctgaaaatgcactgaatatttttggagggtttactggggtgccagacgccactttaggagttttacatatcctctccatttcatttagttctcttagcacagagaagtgggagaagatagtcccattttacaggtgggatgaagagagagatggaggaatttgccccaggttactcagctagaaggtggtgaagaactcaagccttcggatatcagcgcctggcatttaactaccaatcggtcctgctgggactccggctcctctggcaccatccccgggacctactcagagagtttgcacgtggccggtcgcgttccatcgtctaacaaggtccagcacagcgcaaatccgaagatcgtctaccccggggaaaaagagagtctgtttaattctcctgtggccctccaagtgagttcttttgggttccattgcctagacgaggaaagtgaggctttgcctgctctgcgctcacagggtcggcaagtagtgggaccctaggttcctgcagtattccagagataatcaaagctgcacaggtctcgtcatttttatgcaaaggcgtccggaaggctcgaactctcccttgcacaagcccatctgtctctgtgcgccgcccccgggacacggaagcaggcggcgagcagcgccgagtgggtggagaaccgtcccccgccactcacccctcggccaactctccgcgccttctcagccggcacccacgaggccgacctctctcggcctaaaaaaaaaaaaaaaaaatcccggcctcccctgcaccccgcccgccgcccccagggagctgcattaatattaatctcgctgaataattgaaggccagagatttattcgagcttcggcgggggagggagcgcagctgggccgcgtttaggctgcaccacccgcgtgtttcagccgctcgactccgctggacctgggacccccagacgtgggaggatggggtgggtgtgcctgcctgtgagtttgggggtgagtgtgagctgaagcgggtgctccggggagtgaggagggagcgccaggggctgctccagggaggcggagacggaggggcatcccgggtctccgcgcggtcgcctgcgcttcaccccgcacggggtgacctggggccacgcgggcttcaggggaaacaatagctactccttagatcctgggctcctgccaccggctgcccaagccttcccggacgagcggcggggcctcttttcttatttggctaatttatggcgagaggctgggggaagggatggcagaggagggaccgcgactgaaaatgggggcggggggcggcggttaaaggagttgcccgaggcggcggcgcgggtgatgtcagctctcgacgaaaatagagagggatcgcctgcaaatccccagctccggcggggctaaaccttgcaatccctccctggccggcgccgagccagagcgcagcggcctccaccgcctccccaggcgcgcacacacccgcacacgcgcacgcacgctcaccgtcctctgccaccactctctgctcccgccactcgccgcgcccgcgagccccgcagcaaagcacaggtggcagcggctgcaggggcgcatcgccggcgtgcgccctcctgcagccctgggcgcatcgctctctcggggaagccaccctcggagcccccggagctccccgccaagcgccatccccgcgggcggaggggagcgcgggtcgcgcgccgtggagagccgggacgcggattagcgcccgcaggagcctcctgcgcccgttgaggcgctaaagggcttaccccggaggcgggtggaagggcgggcagaggctcctcttaaataccgctcccggccgcacttcgcgctcaccccggcgtccgctttctccctcgcccacagctgccggatagtgctgaagaggagggggcgttccccagaccatgg

SEQ ID NO: 3 - DNA, 2454 bp, Homo sapiens
ggtaccttgctcttggcagtgagcgtgaatgaatggcacagctcagcccctgaagcctgtgtgcagagattgagggattgtgatggagtagttcattcatgctcatgttaaggggggtgctaatagcagactagtgctcctgcgattattaatatctaggtctgggacagattgtgatggcttcttttccagttgccacctcagcagaaagggaaatagaaaaccctaacttgtaaagttagacaattagactgtaaagtttgtatatgtgacaacttcagatacaaagacacacacttacccttgacggggcttaagaggagagtgtcaaacataataccaaagtgaaagaagatagctcttcatctacaaattatttttaaacacatttaccaggttaaacaataactaatttttcggaagagaagagtacccaaagtcaaatgccctaagacgaagagatgcttatggcatttttttttaaataaagaaaatgcaaagttagagtggttctgaaggaacctaggatgaataaggtacagacatgattattctaatggtgcagacaggattgagagagaaggggggaggggagagatggagaaaggcatggatggaagatgacgtttggattcagattttggaaaggagagtaaaggaaggaggtaagcagagatttattttttaaattttattaatgtgttttcccctctttttcttgttatttttctcatctgtctgttcatacttggatattttgtccaataaactatcttctaaggactctgaaaatgcactgaatatttttggagggtttactggggtgccagacgccactttaggagttttacatatcctctccatttcatttagttctcttagcacagagaagtgggagaagatagtcccattttacaggtgggatgaagagagagatggaggaatttgccccaggttactcagctagaaggtggtgaagaactcaagccttcggatatcagcgcctggcatttaactaccaatcggtcctgctgggactccggctcctctggcaccatccccgggacctactcagagagtttgcacgtggccggtcgcgttccatcgtctaacaaggtccagcacagcgcaaatccgaagatcgtctaccccggggaaaaagagagtctgtttaattctcctgtggccctccaagtgagttcttttgggttccattgcctagacgaggaaagtgaggctttgcctgctctgcgctcacagggtcggcaagtagtgggaccctaggttcctgcagtattccagagataatcaaagctgcacaggtctcgtcatttttatgcaaaggcgtccggaaggctcgaactctcccttgcacaagcccatctgtctctgtgcgccgcccccgggacacggaagcaggcggcgagcagcgccgagtgggtggagaaccgtcccccgccactcacccctcggccaactctccgcgccttctcagccggcacccacgaggccgacctctctcggcctaaaaaaaaaaaaaaaaaatcccggcctcccctgcaccccgcccgccgcccccagggagctgcattaatattaatctcgctgaataattgaaggccagagatttattcgagcttcggcgggggagggagcgcagctgggccgcgtttaggctgcaccacccgcgtgtttcagccgctcgactccgctggacctgggacccccagacgtgggaggatggggtgggtgtgcctgcctgtgagtttgggggtgagtgtgagctgaagcgggtgctccggggagtgaggagggagcgccaggggctgctccagggaggcggagacggaggggcatcccgggtctccgcgcggtcgcctgcgcttcaccccgcacggggtgacctggggccacgcgggcttcaggggaaacaatagctactccttagatcctgggctcctgccaccggctgcccaagccttcccggacgagcggcggggcctcttttcttatttggctaatttatggcgagaggctgggggaagggatggcagaggagggaccgcgactgaaaatgggggcggggggcggcggttaaaggagttgcccgaggcggcggcgcgggtgatgtcagctctcgacgaaaatagagagggatcgcctgcaaatccccagctccggcggggctaaaccttgcaatccctccctggccggcgccgagccagagcgcagcggcctccaccgcctccccaggcgcgcacacacccgcacacgcgcacgcacgctcaccgtcctctgccaccactctctgctcccgccactcgccgcgcccgcgagccccgcagcaaagcacaggtggcagcggctgcaggggcgcatcgccggcgtgcgccctcctgcagccctgggcgcatcgctctctcggggaagccaccctcggagcccccggagctc

SEQ ID NO: 4 - DNA, 861 bp, Homo sapiens
cccgggtctccgcgcggtcgcctgcgcttcaccccgcacggggtgacctggggccacgcgggcttcaggggaaacaatagctactccttagatcctgggctcctgccaccggctgcccaagccttcccggacgagcggcggggcctcttttcttatttggctaatttatggcgagaggctgggggaagggatggcagaggagggaccgcgactgaaaatgggggcggggggcggcggttaaaggagttgcccgaggcggcggcgcgggtgatgtcagctctcgacgaaaatagagagggatcgcctgcaaatccccagctccggcggggctaaaccttgcaatccctccctggccggcgccgagccagagcgcagcggcctccaccgcctccccaggcgcgcacacacccgcacacgcgcacgcacgctcaccgtcctctgccaccactctctgctcccgccactcgccgcgcccgcgagccccgcagcaaagcacaggtggcagcggctgcaggggcgcatcgccggcgtgcgccctcctgcagccctgggcgcatcgctctctcggggaagccaccctcggagcccccggagctccccgccaagcgccatccccgcgggcggaggggagcgcgggtcgcgcgccgtggagagccgggacgcggattagcgcccgcaggagcctcctgcgcccgttgaggcgctaaagggcttaccccggaggcgggtggaagggcgggcagaggctcctcttaaataccgctcccggccgcacttcgcgctcaccccggcgtccgctttctccctcgcccacagctgccggatagtgctgaagaggagggggcgttccccagaccatgg

(19) United States
(12) Patent Application Publication
(10) Pub. No.: US 2007/0238717 A1
(43) Pub. Date: Oct. 11, 2007

(54) Neuroprotection with Beta-Lactam Compounds

(75) Inventors: Jeffrey D. Rothstein, Catonsville, MD (US); Dorothy Chung, Potomac, MD (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Johns Hopkins University, Baltimore, MD (US)

(21) Appl. No.: 10/576,670

(22) PCT Filed: Oct. 21, 2004
(86) PCT No.: PCT/US04/35011

§ 371 Date: Feb. 12, 2007

Related U.S. Application Data
(60) Provisional application No. 60/513,037, filed on Oct. 21, 2003.
(60) Provisional application No. 60/541,589, filed on Feb. 4, 2004.

(51) Int. Cl. A61K 31/545; A61K 31/546; G01N 33/53; G01N 33/561

(52) U.S. Cl. 514/206; 435/7.1; 436/516; 514/200

(57) ABSTRACT

Described herein are methods of treating or preventing neurological disease and disease symptoms using beta-lactam compounds, and compounds useful for treating disease and disease symptoms.

### RELATED APPLICATIONS

This application claims the benefit of U.S. Provisional Application Ser. Nos. 60/513,037, filed Oct. 21, 2003, and 60/541,589, filed Feb. 4, 2004. The entire contents of these provisional applications is incorporated herein by this reference.

### GOVERNMENT SUPPORT

This work described herein was supported by a grant from the National Institutes of Health (Grant No. NS33958). Therefore, the U.S. Government may have certain rights in the invention.

### BACKGROUND OF THE INVENTION

Neurological disorders can significantly impact the central nervous system (CNS) and motor neuron units. For example, certain neurological disorders of the CNS are known to adversely affect the brain and associated structures. Neurological disorders affecting motor neuron units have been grouped into motor neuron diseases and peripheral neuropathies. See generally Kandel, E. R. et al; (1991) in Principles of Neuroscience, Appleton & Lange, Norwalk, Conn.; and Rowland, L. P. (ed.) (1982) in Human Motor Neuron Diseases. New York. Raven Press.

An illustrative motor neuron disease is amyotrophic lateral sclerosis (ALS). ALS has been reported to be a chronic neuromuscular disorder having recognized clinical manifestations. For example, it has been suggested that degeneration of cortical and spinal/bulbar motor neurons may play a key role in the disorder. ALS is nearly always fatal. About 95% of all ALS cases are sporadic, with many of the remaining cases showing autosomal dominant inheritance. See e.g., Kuncl R. W. et al., (1992) Motor Neuron Diseases In Diseases of the Nervous System, Asbury et al. eds. (Philadelphia W.B. Saunders) pp. 1179-1208; Brown, R. H., (1996) Amer. Neurol. 30:145; Siddique, T. and Deng., H. X. (1996) Hum. Mol. Genetics. 5:1465).

Specific CNS disorders have been also described. In particular, some have been attributed to cholinergic, dopaminergic, adrenergic, serotonergic deficiencies or combinations thereof CNS disorders of severe impact include pre-senile dementia (sometimes referred to as Alzheimer's disease (AD) or early-onset Alzheimer's disease), senile dementia (dementia of the Alzheimer's type), Parkinson's disease (PD), and Huntington's disease (HD, sometimes referenced as Huntington's chorea). Such CNS disorders are well-represented in the human population. See generally; Gusella, J. F. et al. (1983) Nature 306: 234; Borlauer. W. and Jprmuloewoca P. (eds.) (1976); Adv. in Parkinsonism: Biochemistry, Physiology, Treatment. Fifth International Symposium on Parkinson's Disease (Vienna) Basel: Roche; and references cited therein.

Significant attention has been directed towards understanding the etiology of motor neuron diseases. For example, abnormal levels of certain excitotoxic neurotransmitters have been reported to adversely contribute to many motor neuron diseases. In particular, glutamate-mediated excitotoxicity is recognized to have a critical role in ALS. See e.g., Rothstein J. D. et al., (1990) Ann. Neurol. 28: 18; Rothstein J. D. et al.(199<2) N. Engl. Med. 326: 1464; Rothstein J. D. et al. (1993) PNAS (USA) 90: 6591; and Lacomblez, L. et al., (1996) Lancet 347: 1179.

The astroglial transporters GLAST (EAAT1) and GLT-1 (EAAT2) are responsible for the largest percentage of glutamate transport in the forebrain. As such, they both represent intriguing targets for modulation of expression, and thereby as agents to retard disease progression, including neurodegeneration, seizure, and brain tumor growth.

There has been substantial efforts towards understanding mechanisms for reducing glutamate levels in the nervous system. For example, high-affinity, sodium-dependent glutamate transport is one reported means of inactivating glutamate. In particular, astrocytic excitatory amino acid transporter 2 (EAAT2) proteins are believed to have substantial functions in that inactivation. See e.g., Rothstein J. D. et al. (1994) Neuron 28: 18; Rothstein J. D. et al., (1995) Ann. Neurol. 38: 78. and references cited therein.

In particular, investigations have suggested that EAAT2 is a predominant glutamate transporter. More particularly, certain antisense knockdown studies have been reported to demonstrate that EAAT2 loss can lead to excitotoxic neuronal degeneration and progressive motor impairment. Studies of ALS and other neurodegenerative disorders have related impaired glutamate transport to loss of the EAAT2 protein. In particular, up to 60% to 70% of the sporadic ALS patients examined have a 30% to 95% loss of the EAAT2 protein. See e.g., Haugeto et al., supra; Rothstein J. D., et al., (1996) Neuron 16: 675; Bristol, L. A. and Rothstein, J. D. (1996) Ann. Neurol. 39: 676.

There have been attempts to treat or prevent neurological disorders of the CNS and the motor neuron units. However, most existing therapies do not always stem the development or severity of the disorders in afflicted patients. See e.g., Rowell, (1987) Adv. Behav. Biol. 31: 191; Rinne, et al. Brain Res. (1991) 54: 167; U.S. Pat. No. 5,210,076 to Berliner; Yurek, D. M. (1990) Ann. Rev. Neurosci. 13: 415, and Rowland et al. supra.

Accordingly, there is a need in the field for effective therapies for treating neurological disorders.

### SUMMARY OF THE INVENTION

Described herein are methods of modulating excitatory amino acid transporter (EAAT) protein expression, methods of treating disease and disease symptoms, and compounds useful for modulating EAAT protein expression and treating disease and disease symptoms.

In one aspect, the invention provides a method of treating a subject suffering from or susceptible to a disease or disorder associated with neurodegeneration. The method includes the step of administering to the subject a therapeutic amount of an amount of a beta-lactam compound sufficient to treat the disease or disorder or symptoms thereof associated with neurodegeneration under conditions such that the disease or disorder associated with neurodegeneration is treated. In certain preferred embodiments, the subject is a human. In certain preferred embodiments, the subject is a subject identified as being in need of such treatment. In certain preferred embodiments, the subject is not suffering from a bacterial infection. In certain preferred embodiments, the step of administering the beta-lactam compound comprises administering the beta-lactam compound for a period of at least about 3 weeks. In certain preferred embodiments, the step of administering the beta-lactam compound comprises administering the beta-lactam compound for a period of at least about 6 months. In certain preferred embodiments, the step of administering the beta-lactam compound comprises administering the beta-lactam compound in a dosage of less than about 250 mg/day. In certain preferred embodiments, the step of administering the beta-lactam compound comprises administering the beta-lactam compound in an amount which does not result in substantial clinically effective antibiotic activity when administered to the subject. In certain preferred embodiments, the average maximum plasma concentration of the beta-lactam compound in the subject does not exceed about 10 micrograms per milliliter.

In certain embodiments, the methods herein are those wherein the subject is a mammal, more preferably a primate, most preferably a human.

In certain preferred embodiments, the beta-lactam compound is a cephalosporin, more preferably ceftriaxone, even more preferably ceftriaxone sodium, and still more preferably ceftriaxone disodium salt, sesquaterhydrate.

In certain preferred embodiments, EAAT2 protein expression is increased in vivo; preferably EAAT2 production is increased by 200% or more relative non-regulated production.

In certain preferred embodiments, the disease or disorder associated with neurodegeneration is selected from the group consisting Parkinson's disease, Huntington's disease, Alzheimer's disease, multiple sclerosis, amyotrophic lateral sclerosis, acute neurological diseases, epilepsy, spinal cord injury, brain trauma, glaucoma, and psychiatric disorders.

In certain preferred embodiments, the step of administering comprises administering the compound intravenously or intramuscularly.

In certain embodiments, the method further includes the step of determining a level of EAAT (e.g., EAAT2) expression in the subject. In certain embodiments, the step of determining of the level of EAAT expression is performed prior to administration of the beta-lactam compound to the subject. In certain embodiments, the determining of the level of EAAT expression is performed subsequent to administration of the beta-lactam compound to the subject. In certain embodiments, the determining of the level of EAAT expression is performed prior to and subsequent to administration of the beta-lactam compound to the subject. In certain embodiments, the levels of EAAT expression performed prior to and subsequent to administration of the beta-lactam compound to the subject are compared. In certain embodiments, the comparison of EAAT levels is reported by a clinic, laboratory, or hospital agent to a health care professional. In certain embodiments, when the level of EAAT expression performed prior to administration of the beta-lactam compound to the subject is lower than the level of EAAT expression performed subsequent to administration of the beta-lactam compound to the subject, then the amount of compound administered to the subject is an effective amount.

In another aspect, the invention provides kits for treatment of a disease(s) or disorder(s) associated with neurodegeneration. In one embodiment, the kit includes an effective neuroprotective amount of a beta-lactam compound in unit dosage form, together with instructions for administering the beta-lactam compound to a subject suffering from or susceptible to a disease or disorder or symptoms thereof associated with neurodegeneration, wherein the effective neuroprotective amount of a beta-lactam compound is less than 250 mg of the compound. In preferred embodiments, the beta-lactam compound is a cephalosporin, cephalosporin, more preferably ceftriaxone, even more preferably ceftriaxone sodium, and still more preferably ceftriaxone disodium salt, sesquaterhydrate.

The methods delineated herein are also those wherein the extracellular glutamate concentration is reduced by at least about 50% relative non-regulated concentration; or those wherein the extracellular glutamate concentration is reduced by at least about 75% relative non-regulated concentration. The method can be that wherein the EAAT2 expression promoting agent is a β-lactam antibiotic; or that wherein the EAAT2 expression promoting agent is a penicillin class, cephalosporin class, carbapenam class or monobactam class compound.

In another aspect, the invention provides a method of treating a mammal to modulate glutamate neurotransmission, the method including administering to the mammal a therapeutically effective amount of at least one EAAT expression promoting agent (e.g., a beta-lactam compound, e.g., ceftriaxone) capable of increasing EAAT2 expression. In other aspects, the methods are those wherein the mammal is in need of treatment for a condition that is associated with learning or memory, or those wherein the administration is for enhancing learning, memory; or cognitive enhancement.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

Table 1 lists compounds (or salts or solvates thereof) useful in the methods delineated herein.

TABLE 1
β-lactam compounds

| | Cephalosporins and | Other Beta |
|---|---|---|
| Penicillins | cephamycins | Lactams |
| benzylpenicillin (penicillin g) | cefaclor | Aztreonam |
| procaine benzylpenicillin | cefadroxil | Imipenem |
| (procaine penicillin) | cefadyl | Meropenem |
| phenoxymethylpenicillin | cefalexin | Ertapenem |
| (penicillin v) | cefamandole | FK-037 |
| benzathine penicillin | cefazolin | |
| hetacillin | cefditoren | |
| cloxacillin | cefepime | |
| carbenicillin | cefetamet | |
| flucloxacillin | cefdinir | |
| ampicillin | cefixime | |
| amoxicillin | cefizox | |
| co-amoxiclav | cefotaxime | |
| carboxypenicillin | cefmetazole | |
| ticarcillin | cefobid | |
| timentin | cefonicid | |
| tazocin (ureidopenicillin | cefoperazone | |
| piperacillin with the beta- | cefotan | |
| lactamase inhibitor tazobactam) | cefotetan | |
| piperacillin | cefoxitin | |
| pivmecillinam | cefpirome | |
| amoxicillin-clavulanate | cefpodoxime | |
| piperacillin | cefpodoxime proxetil | |
| oxacillin | cefprozil | |
| | cefradine | |
| | ceftazidime | |
| | ceftibuten | |
| | ceftidoren | |
| | ceftin | |
| | ceftizoxime | |
| | ceftriaxone | |
| | cefuroxime | |
| | cefuroxime axetil | |
| | cephalexin | |
| | cefzil | |
| | cephalothin | |

### BRIEF DESCRIPTION OF THE DRAWINGS

FIG. 1 is sequence listings for SEQ ID NOs: 1-4, which are EAAT2 promoter sequences.

FIG. 2A shows spinal cord cultures incubated with test compound; 2B is a sample slot blot from tissue homogenates; 2C illustrates a representative screening slot blot; 2D illustrates screening results of a library of test compounds; 2E is an illustration of expression results from treatment with various compounds categorized by classes; 2F shows a dose-response analysis of EAAT2 expression for ceftriaxone.

FIG. 3A-3E illustrate expression of EAAT2 promoter fragments in mouse brain; 3F shows astrocytes from EAAT1 promoter reporter, and 3G cortical expression, in transgenic mice.

FIG. 4A shows activation (by compound class) of EAAT2 promoter by various test compounds; 4B illustrates dose-response results.

FIG. 5A is a western blot of ceftriaxone effect on GLT-1 and GLT-1B expression; 5B illustrates the effect of ceftriaxone on GLT-1 and GLT-1B expression; 5C is a western blot of ceftriaxone effect on GLAST, EAAC1 and EAAT4 expression; 5D illustrates the effect of ceftriaxone on GLAST, EAAC1 and EAAT4 expression.

FIG. 6 illustrates the effect of various antibiotics on glutamate transport.

FIG. 7A illustrates the effect of ceftriaxone on ischemic tolerance; 7B illustrates the effect of ceftriaxone on motor neuron degeneration; 7C illustrates the effect of ceftriaxone on grip strength (in vivo model); 7D illustrates the effect of ceftriaxone on survival in G93A mice.

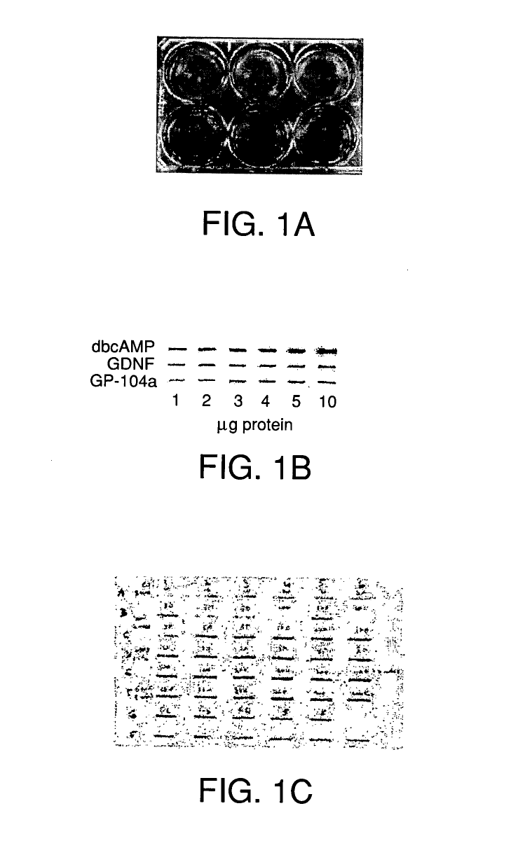

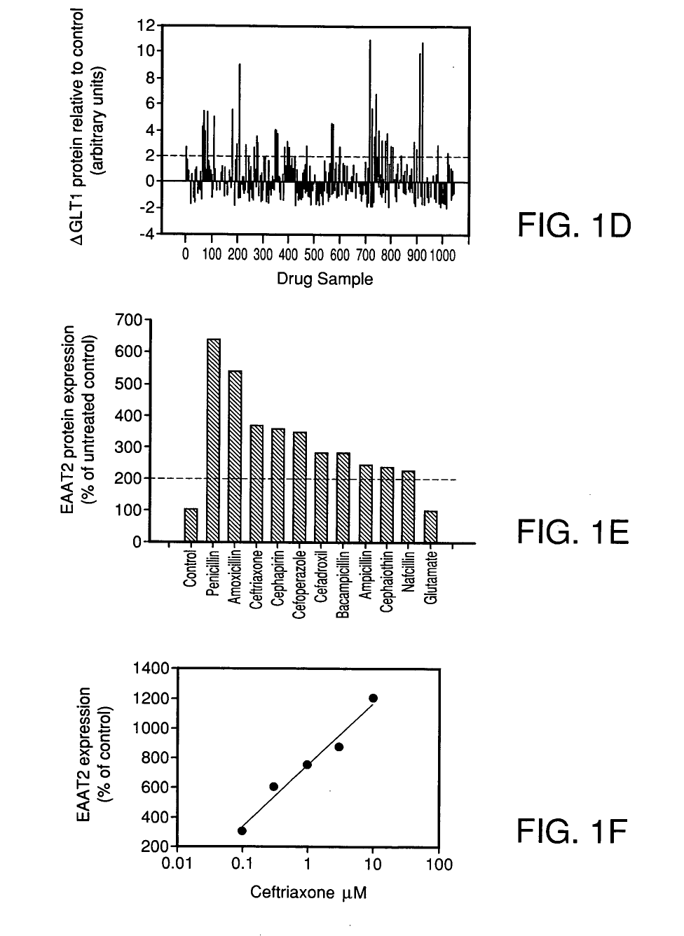

### DETAILED DESCRIPTION OF THE INVENTION

The acidic amino acids glutamate (Glu) and aspartate are the predominant excitatory neurotransmitters in the mammalian central nervous system (CNS). Although there are millimolar concentrations of these excitatory amino acids (EAAs) in the brain, extracellular concentrations are maintained in the low micromolar range to facilitate crisp synaptic transmission and to limit the neurotoxic potential of these EAAs. A family of Na⁺-dependent high affinity transporters is responsible for the regulation and clearance of extracellular EAAs.

Glutamate and aspartate activate ligand-gated ion channels that are named for the agonists N-methyl-D-aspartate (NMDA), α-amino-3-hydroxy-5-methyl-4-isoxazolepropionate (AMPA), and kainate. These ionotropic EAA receptors mediate rapid synaptic depolarization and are important for a number of other physiological processes, including synaptic plasticity and synapse development. The EAAs also activate a family of metabotropic receptors coupled through G-proteins to second messenger systems or ion channels. It is well established that the EAAs are extremely important for normal brain function. However, there is substantial evidence that an extracellular accumulation of EAAs and excessive activation of EAA receptors also contributes to the neuronal cell death observed in acute insults to the CNS. The process known as, ‘excitotoxicity’, may also contribute to neuronal loss observed in chronic neurodegenerative diseases, including amyotrophic lateral sclerosis (ALS).

The intracellular concentrations of glutamate (5-10 mM) and aspartate (1-5 mM) are 1000-fold to 10,000-fold greater than the extracellular concentrations (<1-10 μM). Unlike many other neurotransmitters, there is no evidence that glutamate or aspartate is metabolized extracellularly. Instead, they are cleared from the extracellular space by transport into neurons and astrocytes.

Several subtypes of Na⁺-dependent glutamate transporters have been identified through pharmacological strategies and cDNA cloning. Five known distinct cDNA clones that express Na⁺-dependent high-affinity glutamate transport are referred to herein as GLT-1/EAAT2, EAAC1/EAAT3, GLAST/EAAT1, EAAT4, and EAAT5. There is also evidence for additional heterogeneity of GLT-1 and GLAST that originates from alternate mRNA splicing.

Expression of two of these transporters, GLT-1 and GLAST, is generally restricted to astroglia Expression of two other transporters, EAAC1 and EAAT4, is generally restricted to neurons, and EAAT5 is thought to be restricted to retina. Of the three transporters found in forebrain (GLT-1, GLAST, and EAAC1), GLT-1 appears to be the only transporter that is specific to brain tissue, suggesting that GLT-1 expression is controlled by brain specific mechanisms.

Previously, it was thought that presynaptic transporters had a major role in the clearance of EAAs during synaptic transmission. This theory was based on evidence which showed that activity is enriched 2-fold in synaptosomal membrane preparations compared to fractions enriched in mitochondria or myelin. However, it is now known that these membrane preparations contain resealed glial membranes and tremendous amounts of GLT-1 protein. In addition, it has long been known that lesions of specific afferents result in a decrease in Na⁺-dependent transport in target areas. For example, lesions of the cortical projections to the striatum result in decreased uptake in striatal synaptosomes. These types of studies suggested that there was significant transport into presynaptic terminals, but more recent studies have suggested that these lesions reduce expression of the glial transporters.

Evidence from several complementary strategies strongly suggests that GLT-1 mediates the bulk of Na⁺-dependent transport of EAAs in the CNS. For example, the pharmacological properties of GLT-1 parallel the predominant component of activity observed in rat brain membranes. Based on the enrichment required to purify GLT-1 to homogeneity, it is thought that GLT-1 represents approximately 1% of total brain protein. Selective immunoprecipitation of GLT-1 from solubilized forebrain tissue and reconstitution of the remaining protein in liposomes, suggests that GLT-1 mediates 90% of transport activity. Anti-sense knock-down of GLT-1 results in the dramatic reductions in synaptosomal transporter activity in several forebrain regions. Synaptosomal uptake in mice genetically deleted of GLT-1 is 5% of normal. Finally, electrophysiological recording of transporter mediated currents in brain preparations strongly suggest that GLT-1 has a primary role for the clearance of glutamate during synaptic transmission in several forebrain regions.

The expression of GLT-1/EAAT2 is dynamically regulated both in vivo and in vitro. Although GLT-1 is the predominant transporter in the adult CNS, expression is rather low early in development and increases during synaptogenesis in both rats and humans. As described above, lesions of projections to a particular target nucleus results in decreased expression of both glial transporters, GLT-1 and GLAST. These data suggest that the presence of neurons induces and/or maintains expression of the glial transporters.

Several different groups have demonstrated decreased expression of GLT-1 and/or GLAST in animal models of acute insults to the CNS, including stroke and traumatic brain injury. A loss in GLT-1 expression has been demonstrated in patients with ALS. Furthermore, there is evidence of decreased expression of these transporters in humans afflicted with chronic neurodegenerative diseases, including Alzheimer's Disease, and Huntington's Disease. Loss of GLT-1 is also a feature of the fatal brain tumor, glioblastoma multiforma.

Amyotrophic lateral sclerosis (ALS) is the most common form of adult motor neuron disease in which there is progressive degeneration of both the upper motor neurons in the cortex and the lower motor neurons in the brain stem and spinal cord. The majority of ALS cases (95%) are apparently sporadic (SALS), while approximately 5% are familial (FALS). Cleveland D W, Rothstein J D., Nat. Rev. Neurosci. (2001); 2:806-819; Kuncl R W, Crawford T O, Rothstein J D, Drachman D B. Motor neuron diseases. In: Asbury A K et al., editors. Diseases of the Nervous System. 2 ed. Philadelphia: W.B. Saunders, 1992:1179-1208. FALS cases were found to be associated with mutations in SOD-1. Andersen P M et al. Genetics of amyotrophic lateral sclerosis: an overview. In: Brown R H, Jr., Meininger V, Swash M, editors. Amyotrophic lateral sclerosis. London: Martin Dunitz, 2000:223-250; Brown R H, Jr. Amyotrophic lateral sclerosis. Insights from genetics. Arch Neurol 1997; 54:1246-1250; Cleveland D W, Rothstein J D. Nat. Rev. Neurosci. 2001; 2:806-819, the gene that encodes copper-zinc superoxide dismutase (CuZnSOD). SOD1 mutations account for about 15-20% of all FALS. SOD1 mutations have been used to generate transgenic mouse models; SOD1 polypeptides with at least one amino acid substitution selected from G93A, G37R, G86R and G85R all produce reliable motor neuron degeneration in transgenic mice over-expressing the mutant protein. Cleveland D W., Neuron 1999; 24:515-520; Cleveland D W et al., Nature 1995; 378:342-343; Cleveland D W, Rothstein J D. Nat. Rev. Neurosci. 2001; 2:806-819. Pathogenic events “downstream” of mutant SOD1 toxicity include excitotoxicity, neuroinflamrmation and apoptosis. Increasingly, these downstream events have been the target of pharmacotherapy, which in some cases can successfully alter disease course. Multiple other genes or chromosomal localization have been identified in other familial variants of ALS.

Common to both familial and sporadic ALS is the loss of the astroglial glutamate transporter EAAT2 protein. As described above, the astroglial transporter EAAT2 is the predominant protein responsible for the bulk of synaptic clearance of glutamate. In particular, EAAT2 protects against excitotoxic neurodegeneration.

Evidence of abnormalities in glutamate handling initially arose in ALS from discovery of large increases in cerebral fluid levels of glutamate in ALS patients; such findings are now reported in ˜40% of sporadic ALS patients. Rothstein J D et al. Ann. Neurol. 1990; 28:18-25; Spreux-Varoquaux O et al. J Neurol Sci. 2002; 193:73-78. Measurement of functional glutamate transport in ALS tissue revealed a marked diminution in the affected ALS brain regions. The loss of functional glutamate transporter is likely the result of a dramatic loss of astroglial glutamate transporter protein EAAT2, which can be in up to 65% of sporadic ALS patients. Rothstein J D et al., Ann. Neurol. 1994; 36:282; Rothstein J D, et al. N Engl. J Med 1992; 326:1464-1468; Rothstein J D et al., Ann Neurol 1995; 38:73-84. Regardless of the mechanism, lowering EAAT2 with antisense oligonucleotides has demonstrated that loss of transport activity directly provokes neuronal death. Furthermore, expression of at least three (G85R, G93A, G37R)SOD1 mutants in transgenic mice—all lead to a loss of the EAAT2 protein and its function. Rothstein J D et al., Neuron 1996; 16:675-686; Rothstein J D et al., Proc Natl Acad Sci USA 1993; 90:6591-6595. In aggregate, these and other studies suggest that that the functional loss of EAAT2 (associated with astrocyte dysfunction), contributes to the loss of motor neurons in both inherited and sporadic ALS. Recently, we also documented a loss of the GLT-1/EAAT2 protein in a new rat transgenic model of the disease. Howland D S et al., Proc. Natl. Acad. Sci. USA 2002; 99:1604-1609. Notably, loss of transporter protein precedes actual degeneration of motor neurons and their axons in the rat model.

Two labs recently provided important data as to the importance of EAAT2 as a therapeutic. Dr. Glen Lin, reported that a 2 fold overexpression of EAAT2, in transgenic mice, leads to neuroprotection in vitro, and delayed onset of disease in ALS mice. Guo, H. et al. Hum Mol. Genet. 2003; 12:2519-2532. Similarly, Dr. Margaret Sutherland, has reported that a five fold over expression of EAAT2 in tansgenic mice, can increase survival of G93A SOD1 mice by at least 30 days (and in several animals many months longer). Maguire J L, et al. Soc Neurosci Abstr. 2001; 27:607.9; Sutherland M. L., et al. Soc Neurosci Abs. 2003. In addition, her lab has reported that increased EAAT2 can also attenuate seizures and significantly diminished both seizures and tumor growth in glioma xenografted rodents. As will be shown below, we have also generated data that suggest that over expression of EAAT2 can delay disease onset in ALS mice, using novel therapeutics.

Even though GLT-1 expression is extremely high in vivo, ‘normal’ astrocytes maintained in culture express essentially no detectable mRNA or protein. Co-culturing astrocytes with neurons induces glial expression of GLT-1, suggesting that neurons induce and/or maintain expression of GLT-1 in vitro. This effect of neurons is, at least in part, mediated by a soluble secreted molecule. Several small molecules mimic this effect of neurons, including dbcAMP, epidermal growth factor, pituitary adenylate cyclase-activating peptide, and immunophilin. In all of these cases the increases in GLT-1 protein expression are accompanied by an increase in GLT-1 mRNA and a change in the morphology of the astrocytes that many believe are reminiscent of differentiation.

The effects of dbcAMP are blocked by an inhibitor of protein kinase A. It has been shown that the increase in GLT-1 expression induced by dbcAMP, epidermal growth factor, or neuron conditioned medium are all blocked by an inhibitor of either phosphatidylinositol 3-kinase or an inhibitor of the transcription factor NF-κB. Otherwise, little is known about the mechanisms that actually control GLT-1 expression. Thus, the identification—of the EAAT2 promoter provides a valuable tool to understand EAAT2 regulation and to develop assays to control its synthesis.

In published PCT patent application no. WO2004/076675, the disclosure of which is incorporated herein by reference in its entirety, we disclosed that beta-lactam compounds, including antibiotics such as ceftriaxone, can increase expression of EAAT2 and can therefore be used to treat diseases and disorders associated with neurodegeneration. We have now discovered that effective neuroprotective doses of these compounds are lower than the amounts commonly administered for antibiotic therapy.

As used herein, the term “neuroprotective” refers to compositions and treatments that are effective to prevent or reduce death or damage to nerve cells, (including neurons and glia), or rescuing, resuscitating or reviving nerve cells, e.g., nerve cells that have sustained damage or injury.

As used herein, the term “EAAT2” refers to the human astroglial glutamate transporter 2 gene. See, e.g., U.S. Pat. No. 5,658,782 which discloses the human EAAT2 cDNA sequence, the disclosure of the which is specifically incorporated herein by reference. As used herein, the term “GLT-1” refers to the rodent astroglial glutamate transporter 2 gene.

As used herein, the term “promoter” generally refers a region of genomic DNA, usually found 5′ to an mRNA transcription start site. Promoters are involved in regulating the timing and level of mRNA transcription and contain, for example, binding sites for cellular proteins such as RNA polymerase and other transcription factors. As used interchangeably herein, the terms “EAAT2 promoter”, “EAAT2 promoter region” and the like include the region of genomic DNA found 5′ to the EAAT2 mRNA transcription start site. In preferred embodiments, the EAAT2 promoter comprises SEQ ID NO:1, 2, 3, or 4, or fragments thereof. When inserted into a promoterless reporter construct, preferred EAAT2 promoter fragments are able to direct transcription of the reporter gene.

In one embodiment, the EAAT2 promoter includes SEQ ID NO:1 (e.g., nucleotides 1-4696 of SEQ ID NO:1). In another embodiment the EAAT2 promoter includes a P1 region, which comprises nucleotides 733-3450 of SEQ ID NO:1 (also set forth as SEQ ID NO:2). In another embodiment, the EAAT2 promoter includes a P2 region, which comprises nucleotides 733-3186 of SEQ ID NO:1 (also set forth as SEQ ID NO:3). In still another embodiment, the EAAT2 promoter includes a P3 region, which comprises nucleotides 2590-3450 of SEQ ID NO:1 (also set forth as SEQ ID NO:4).

The EAAT2 promoter activation molecules of the present invention provide therapeutic agents for neurological and psychiatric disorders. As used herein, the term ‘neurological disorder’ includes a disorder, disease or condition which affects the nervous system, e.g., the central nervous system. The neurological disorders that can be treated in accord with the present invention include specific disorders that have been reported to be associated with excitotoxicity. Particularly included are specified neurological disorders affecting motor neuron function. Neurological disorders include, but are not limited to, amyotrophic lateral sclerosis (ALS), trinucleotide repeat expansion disorders (e.g., Huntington's disease (HD), spinal and bulbar muscular atrophy, spinocerebellar ataxia types 1, 2, 6, and 7, dentatorubropallidoluysian atrophy, and Machado-Joseph disease), α-synucleinopathies (e.g., Parkinson's disease (PD), dementia with Lewy bodies (DLB), and multiple system atrophy (MSA)), multiple sclerosis (MS), Alzheimer's disease, brain tumors (e.g., glioblastoma), stroke/ischemia, cerebrovascular disease, epilepsy (e.g., temporal lobe epilepsy), HIV-associated dementia, Korsakoff's disease, chronic pain, neurogenic pain, painful neuropathies, peripheral neuropathy, headaches (e.g., migraine headaches), Pick's disease, progressive supranuclear palsy, Creutzfeldt-Jakob disease, Bell's Palsy, aphasia, sleep disorders, glaucoma, and Meniere's disease.

In addition, the EAAT2 promoter activation molecules of the present invention provide therapeutic agents for modulation of normal glutamate neurotransmission associated with brain functions such as learning and memory. The molecules described herein can be administered to a subject in need of such treatment for the enhancement of memory and learning.

As used herein, the term ‘psychiatric disorder’ refers diseases and disorders of the mind, and includes diseases and disorders listed in the Diagnostic and Statistical Manual of Mental Disorders—Fourth Edition (DSM-IV), published by the American Psychiatric Association, Washington D.C. (1994). Psychiatric disorders include, but are not limited to, anxiety disorders (e.g., acute stress disorder agoraphobia, generalized anxiety disorder, obsessive-compulsive disorder, panic disorder, posttraumatic stress disorder, separation anxiety disorder, social phobia, and specific phobia), childhood disorders, (e.g., attention-deficit/hyperactivity disorder, conduct disorder, and oppositional defiant disorder), eating disorders (e.g., anorexia nervosa and bulimia nervosa), mood disorders (e.g., depression, bipolar disorder, cyclothymic disorder, dysthymic disorder, and major depressive disorder), personality disorders (e.g., antisocial personality disorder, avoidant personality disorder, borderline personality disorder, dependent personality disorder, histrionic personality disorder, narcissistic personality disorder, obsessive-compulsive personality disorder, paranoid personality disorder, schizoid personality disorder, and schizotypal personality disorder), psychotic disorders (e.g., brief psychotic disorder, delusional disorder, schizoaffective disorder, schizophreniform disorder, schizophrenia, and shared psychotic disorder), substance-related disorders (e.g., alcohol dependence, amphetamine dependence, cannabis dependence, cocaine dependence, hallucinogen dependence, inhalant dependence, nicotine dependence, opioid dependence, phencyclidine dependence, and sedative dependence), adjustment disorder, autism, delirium, dementia, multi-infarct dementia, learning and memory disorders (e.g., amnesia and age-related memory loss), and Tourette's disorder.

As noted, neurological and psychiatric disorders of specific interest include those associated with abnormal release or removal of excitotoxic amino acids such as glutamate. Several CNS neuron types are especially adversely affected by excitotoxic glutamate. See e.g., Choi, D. W. (1988) Neuron 1: 623; and references cited therein. Specifically preferred neurological disorders include AD, HD, PD with ALS being especially preferred.

III. Methods of Treatment

In one embodiment, the present invention provides methods of treating neurological and/or psychiatric disorders which comprise administering a therapeutically effective amount of a pharmaceutical composition comprising an EAAT2 promoter modulator a subject (e.g., a mammal such as a human). Thus, in one embodiment, the invention provides a method of treating a subject suffering from or susceptible to a disease or disorder associated with neurodegeneration. The method includes the step of administering to the mammal a therapeutic amount of an amount of a beta-lactam compound sufficient to treat the disease or disorder associated with neurodegeneration, under conditions such that the disease or disorder associated with neurodegeneration is treated.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

The preferred therapeutic methods of the invention (which include prophylactic treatment) in general comprise administration of a therapeutically effective amount of an EAAT2 promoter modulator, such as a beta-lactam compound such as ceftriaxone) to an animal in need thereof, including a mammal, particularly a human. Such treatment will be suitably administered to subjects, particularly humans, suffering from, having, susceptible to, or at risk for a neurological or psychiatric disorder. The EAAT2 promoter modulators of the invention may be also used in the treatment of any other disorders in which EAAT2 may be implicated.

For therapeutic applications, EAAT2 modulators of the invention may be suitably administered to a subject such as a mammal, particularly a human, alone or as part of a pharmaceutical composition, comprising the EAAT2 modulator together with one or more acceptable carriers thereof and optionally other therapeutic ingredients. The carrier(s) must be “acceptable” in the sense of being compatible with the other ingredients of the formulation and not deleterious to the recipient thereof.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the beta-lactam compound is administered transdermally (e.g., using a transdermal patch or iontophoretic techniques). Ceftriaxone sodium, sold under the trade name ROCEPHIN, is available as a powder for reconstitution for intravenous or intramuscular administration. Other formulations may conveniently be presented in unit dosage form, e.g., tablets and sustained release capsules, and in liposomes, and may be prepared by any methods well know in the art of pharmacy. See, for example, Remington's Pharmaceutical Sciences, Mack Publishing Company, Philadelphia, Pa. (17th ed. 1985).

Such preparative methods include the step of bringing into association with the molecule to be administered ingredients such as the carrier which constitutes one or more accessory ingredients. In general, the compositions are prepared by uniformly and intimately bringing into association the active ingredients with liquid carriers, liposomes or finely divided solid carriers or both, and then if necessary shaping the product.

In certain preferred embodiments, the bet-lactam compound is administered orally. Compositions of the present invention suitable for oral administration may be presented as discrete units such as capsules, sachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion, or packed in liposomes and as a bolus, etc. Certain formulations have been investigated to increase the oral bioavailability of ceftriaxone (see, e.g., Beskid G et al., Chemotherapy (1988), 34(2):77-84; and U.S. Pat. No. 6,248,360).

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface-active or dispersing agent. Molded tablets may be made by molding in a suitable machine a mixture of the powdered compound moistened with an inert liquid diluent. The tablets optionally may be coated or scored and may be formulated so as to provide slow or controlled release of the active ingredient therein. Methods of formulating such slow or controlled release compositions of pharmaceutically active ingredients, such as beta-lactam antibiotics and other compounds described herein, are known in the art and described in several issued US patents, some of which include, but are not limited to, U.S. Pat. Nos. 3,870,790; 4,226,859; 4,369,172; 4,842,866 and 5,705,190, the disclosures of which are incorporated herein by reference in their entireties. Coatings can be used for delivery of compounds to the intestine (see, e.g., U.S. Pat. Nos. 6,638,534, 5,541,171, 5,217,720, and 6,569,457, and references cited therein).

A skilled artisan will recognize that in addition to tablets, other dosage forms can be formulated to provide slow or controlled release of the active ingredient. Such dosage forms include, but are not limited to, capsules, granulations and gel-caps.

Compositions suitable for topical administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

In preferred embodiments, an EAAT2 promoter activation molecule (such as a beta-lactam compound) is provided in an orally-administered extended-release dosage form. In preferred embodiments, the beta-lactam compound is capable of crossing the blood-brain barrier in therapeutically-effective amounts, i.e., the beta-lactam compound can readily penetrate into the central nervous system.

Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access. Where an organ or tissue is accessible because of removal from the patient, such organ or tissue may be bathed in a medium containing the subject compositions, the subject compositions may be painted onto the organ, or may be applied in any convenient way.

As used herein, the terms “EAAT2 promoter activation molecule” and “beta-lactam compound” include pharmaceutically acceptable derivatives or prodrugs thereof. A “pharmaceutically acceptable derivative or prodrug” means any pharmaceutically acceptable salt, ester, salt of an ester, or other derivative of a compound of this invention which, upon administration to a recipient, is capable of providing (directly or indirectly) an active compound of this invention. Particularly favored derivatives and prodrugs are those that increase the bioavailability of the compounds of this invention when such compounds are administered to a mammal (e.g., by allowing an orally administered compound to be more readily absorbed into the blood) or which enhance delivery of the parent compound to a biological compartment (e.g., the brain or central nervous system) relative to the parent species. Preferred prodrugs include derivatives where a group which enhances aqueous solubility or active transport through the gut membrane is appended to the structure of formulae described herein. See, e.g., Alexander, J. et al. Journal of Medicinal Chemistry 1988, 31, 318-322; Bundgaard, H. Design of Prodrugs; Elsevier: Amsterdam, 1985; pp 1-92; Bundgaard, H.; Nielsen, N. M. Journal of Medicinal Chemistry 1987, 30, 451-454; Bundgaard, H. A Textbook of Drug Design and Development; Harwood Academic Publ.: Switzerland, 1991; pp 113-191; Digenis, G. A. et al. Handbook of Experimental Pharmacology 1975, 28, 86-112; Friis, G. J.; Bundgaard, H. A Textbook of Drug Design and Development, 2 ed.; Overseas Publ.: Amsterdam, 1996; pp 351-385; Pitman, I. H. Medicinal Research Reviews 1981, 1, 189-214.

The compounds of this invention may be modified by appending appropriate functionalities to enhance selective biological properties. Such modifications are known in the art and include those which increase biological penetration into a given biological compartment (e.g., central nervous system), increase oral availability, increase solubility to allow administration by injection, alter metabolism and alter rate of excretion. It will be appreciated that actual preferred amounts of a given EAAT2 modulator of the invention used in a given therapy will vary according to the particular active compound being utilized, the particular compositions formulated, the mode of application, the particular site of administration, the patient's weight, general health, sex, etc., the particular indication being treated, etc. and other such factors that are recognized by those skilled in the art including the attendant physician or veterinarian. Optimal administration rates for a given protocol of administration can be readily determined by those skilled in the art using conventional dosage determination tests, or by any method known in the art or disclosed herein.

For example, in certain embodiments, of this invention, a level of EAAT2 expression or activity in a subject is determined at least once. Comparison of EAAT2 levels, e.g., to another measurement of EAAT2 level obtained previously or subsequently from the same patient, another patient, or a normal subject, may be useful in determining whether therapy according to the invention is having the desired effect, and thereby permitting adjustment of dosage levels as appropriate. Determination of EAAT2 expression levels may be performed using any suitable sampling/expression assay method known in the art or described herein. Preferably, a tissue or fluid sample is first removed from a subject. Examples of suitable samples include blood, mouth or cheek cells, and hair samples containing roots. Other suitable samples would be known to the person skilled in the art. Determination of protein levels and/or mRNA levels (e.g., EAAT2 levels) in the sample can be performed using any suitable technique known in the art, including, but not limited to, ELISA, blotting/chemiluminescence methods, real-time PCR, and the like.

Certain compounds of the invention, such as cephalosporin antibiotics, are conventionally administered for the treatment or prevention of bacterial infection. For example, ceftriaxone is indicated for antibiotic prophylaxis in adults, as treatment of active infections in adults and children (including otitis media and bacterial meningitis in children). In certain embodiments of the present invention, the compounds are administered for prophylaxis or treatment of diseases or disorders associated with neurodegeneration, and in preferred embodiments, the subject is not known to be suffering from a bacterial infection. Thus, in certain preferred embodiments, the subject has not been diagnosed with an active bacterial infection. In certain preferred embodiments, the subject has not been diagnosed as being unusually susceptible to bacterial infection. In other embodiments, the subject has not been diagnosed as having an active bacterial infection nor has the subject been diagnosed as being unusually susceptible to a bacterial infection; however, the subject has been determined to have or to be at risk of developing a neurological disorder or a psychiatric disorder.

Advantageously, the beta-lactam compounds of the invention can be administered for neuroprotection at dosage levels lower than the dosages required for antibiotic therapy. By lowering the amount of beta-lactam compound required for therapy, the cost of treatment is decreased, and potential side effects are alleviated. Such dosage levels are also additionally advantageous in that the subject being administered the beta-lactam compound will have decreased susceptibility to developing bacteria and bacterial infection that is resistant to the beta-lactam compounds being administered, including ceftriaxone, (reference to which includes ceftriaxone sodium, and Rocephin® (ceftriaxone, disodium salt, sesquaterhydrate).

Therefore, in preferred embodiments, compounds of the invention, such as ceftriaxone, are administered at dosage levels lower than the level established for antibiotic activity of the compound (if any). For example, ceftriaxone sodium is typically administered, for antibiotic therapies, in doses of 0.5 to 4.0 grams intravenously or intramuscularly once per day (or twice per day in divided doses) for adults. For pediatric applications, the antibiotic dose is typically 50-75 mg/kg (i.e., about 0.5-0.75 g/day for a 10 kg child), with daily doses not exceeding 2 grams. Thus, in preferred embodiments of this invention, a compound herein, such as ceftriaxone, is administered at a dosage of less than about 500 mg/day, more preferably less than about 250 mg/day, less than about 200 mg/day, less than about 150 mg/day, and still more preferably less than about 100 mg/day, less than about 90 mg/day, less than about 80 mg/day, less than about 70 mg/day, less than about 60 mg/day, less than about 50 mg/day, less than about 40 mg/day, less than about 30 mg/day, less than about 2 mg/day, less than about 15 mg/day, less than about 10 mg/day, less than about 9 mg/day, less than about 8 mg/day, less than about 7 mg/day, less than about 6 mg/day, less than about 5 mg/day, less than about 4 mg/day, less than about 3 mg/day, less than about 2 mg/day, less than about 1 mg/day, less than about 0.9 mg/day, less than about 0.8 mg/day, less than about 0.7 mg/day, less than about 0.6 mg/day, less than about 0.5 mg/day, less than about 0.4 mg/day, less than about 0.3 mg/day, less than about 0.2 mg/day, less than about 0.1 mg/day; or any dosage range in which the low end of the range is any amount between 0.1 mg/day and 400 mg/day and the upper end of the range is any amount between 1 mg/day and 500 mg/day (e.g., 5 mg/day and 100 mg/day, 150 mg/day and 500 mg/day). In other preferred embodiments, a compound herein, such as ceftriaxone, is administered at a dosage of between 0.1 and 100 mg/kg/day, more preferably less than about 50 mg/kg/day, more preferably less than about 40 mg/kg/day, more preferably less than about 30 mg/kg/day, more preferably less than about 25 mg/kg/day, more preferably less than about 20 mg/kg/day, more preferably less than about 15 mg/kg/day, and still more preferably less than about 10 mg/kg/day, less than about 9 mg/kg/day, 8 mg/kg/day, less than about 7 mg/kg/day, less than about 6 mg/kg/day, 5 mg/kg/day, 4 mg/kg/day, 3 mg/kg/day, 2 mg/kg/day, 1 mg/kg/day, 0.9 mg/kg/day, 0.8 mg/kg/day, 0.7 mg/kg/day, 0.6 mg/kg/day, 0.5 mg/kg/day, 0.4 mg/kg/day, 0.3 mg/kg/day, 0.2 mg/kg/day, or 0.1 mg/kg/day; or any dosage range in which the low end of the range is any amount between 0.1 mg/kg/day and 90 mg/kg/day and the upper end of the range is any amount between 1 mg/kg/day and 100 mg/kg/day (e.g., 0.5 mg/kg/day and 2 mg/kg/day, 5 mg/kg/day and 20 mg/kg/day). In certain preferred embodiments, the amount of beta-lactam compound (such as ceftriaxone) administered is less than a clinically effective antibiotic amount. As used herein, the term “clinically effective antibiotic amount” refers to an amount of ceftriaxone (or other antibiotic) that has clinically useful activity in treatment or prevention of bacterial growth in a subject (preferably a human), as determined by standard in vitro or in vivo assays. In certain preferred embodiments, the daily dosage of the beta-lactam compound administered to a subject is less than the daily dosage prescribed to treat a bacterial infection.

In certain embodiments, the compound, e.g., the beta-lactam compound is administered once daily; in other embodiments, the compound, e.g., the beta-lactam compound is administered twice daily; in yet other embodiments, the compound, e.g., the beta-lactam compound is administered once every two days, once every three days, once every four days, once every five days, once every six days, once every seven days, once every two weeks, once every three weeks, once every four weeks, once every two months, once every six months, or once per year. The dosing interval can be adjusted according to the needs of individual patients. For longer intervals of administration, extended release or depot formulations can be used.

As noted above, the compounds of the invention are believed to have neuroprotective activity at levels lower than the levels typically used for antibiotic therapy. Therefore, in preferred embodiments, compounds of the invention, such as ceftriaxone, are administered to achieve levels in plasma (or cerebrospinal fluid (CSF), or other bodily fluids or organs) which are lower than the level established for antibiotic activity of the compound (if any). For example, the compound, e.g., ceftriaxone sodium, can be administered intravenously, for antibiotic therapies, in doses that will achieve a maximum average plasma level of about 82-257 μg/ml (after 0.5 hours). In certain preferred embodiments of this invention, compound, e.g., ceftriaxone plasma levels in a subject being treated according to this invention will be less than about 100 μg/ml, more preferably less than about 90 μg/ml, 80 μg/ml, 70 μg/ml, 60 μg/ml, 50 μg/ml, and still more preferably less than about 40 μg/ml, 30 μg/ml, 20 μg/ml, 10 μg/ml, 5 μg/ml, 4 μl/ml, 3 μg/ml, 2 μg/ml, 1 μg/ml, 0.9 μg/ml, 0.8 μg/ml, 0.7 μg/ml, 0.6 μg/ml, 0.5 μg/ml, 0.4 μg/ml, 0.3 μg/ml, 0.2 μg/ml, or 0.1 μg/ml. In certain preferred embodiments of this invention, compound, e.g., ceftriaxone plasma levels in a subject being treated according to this invention will be less than about 100 μM, more preferably less than about 90 μM, 80 μM, 70 μM, 60 μM, 50 μM, and still more preferably less than about 40 μM, 30 μM, 20 μM, 10 μM, 5 μM, 4 μM, 3 μM, 2 μM, 1 μM, 0.9 μM, 0.8 μM, 0.7 μM, 0.6 μM, 0.5 μM, 0.4 μM, 0.3 μM, 0.2 μM, or 0.1 μM.

Similarly, antibiotics, e.g., ceftriaxone sodium can be administered intravenously, for treatment of pediatric bacterial meningitis, in doses which will achieve a maximum CSF level in inflamed meninges of about 1.3-44 μg/ml (after 3-4 hours). Thus, in preferred embodiments of this invention, ceftriaxone CSF levels in a subject being treated according to this invention will be less than about 50 μg/ml, more preferably less than about 40 μg/ml, 30 μg/ml, 20 μg/ml, or 10 μg/ml, and still more preferably less than about 9 μg/ml, 8 μg/ml, 7 μg/ml, 6 μg/ml, 5 μg/ml, 4 μg/ml, 3 μg/ml, 2 μg/ml, 1 μg/ml, 0.9 μg/ml, 0.8 μg/ml, 0.7 μg/ml, 0.6 μg/ml, 0.5 μg/ml, 0.4 μg/ml, 0.3 μg/ml, 0.2 μg/ml, or 0.1 μg/ml. In certain preferred embodiments of this invention, ceftriaxone CSF levels in a subject being treated according to this invention will be less than about 50 μM, more preferably less than about 40 μM, 30 μM, 20 μM, or 10 μM, and still more preferably less than about 9 μM, 8 μM, 7 μM, 6 μM, 5 μg/ml, 4 μM, 3 X, 2 μM, 1 μM, 0.9 μM, 0.8 μM, 0.7 μM, 0.6 μM, 0.5 μM, 0.4 μM, 0.3 M, 0.2 μM, or 0.1 μM. In certain preferred embodiments, the subject is tested and monitored periodically to determine the level of beta-lactam compound present in the plasma, CSF, or other bodily fluid or tissue, and the dosage adjusted, if required, based on the results of the testing.

It will be appreciated that other active pharmacological agents may be used in combination with beta-lactam compounds in the methods, compositions, and kits of this invention. For example, non-steroidal anti-inflammatory compounds such as aspirin (or other salicylates), naproxen, sulindac, diclofenac, and ibuprofen, and COX-2 inhibitors such as celecoxib (sold under the trade name CELEBREX) and valdecoxib (sold under the trade name BEXTRA), may be used in combination with beta-lactams, e.g., to provide pain relief, reduce inflammation, or provide to provide additive or synergistic benefits for treatment of neurodegenerative diseases or disorders or symptoms thereof. COX-2 inhibitors may be useful for treatment of ALS (see, e.g., Drachman D B, Rothstein J D, Ann Neurol. 2000 48(5):792-5), and in one embodiment of the invention, the combination of a COX-2 inhibitor (preferably celecoxib) with a beta-lactam(s) is provided for treatment of neurodegenerative disorders including ALS. Other exemplary combinations include: a beta-lactam with riluzole (e.g., for treatment of ALS); a beta-lactam with levodopa and/or dopa agonists (e.g., for treatment of Parkinson's disease); a beta-lactam with an acetylcholinesterase inhibitor such as donepezil (sold under the trade name ARICEPT) and/or an NMDA receptor blocker such as memantine (available under the trade name NAMENDA) for treatment of Parkinson's disease; a beta-lactam with gabapentin (available under the trade name NEURONTIN) or amytriptyline for treatment of peripheral neuropathy; or a beta-lactam with an interferon for treatment of multiple sclerosis. Such pharmacological agents, when used in combination with a beta-lactam according to the invention, will be administered by such routes and in such doses as may be determined to be appropriate by one of ordinary skill in the art. Such additional agents may be administered simultaneously with the beta-lactam; may be combined in a single composition with the beta-lactam or may be administered as separate compositions; and may be used for the full duration of beta-lactam therapy or for only certain selected time periods during the beta-lactam therapy.

The compounds of the invention can be used to treat diseases and disease conditions that are acute, and may also be used for treatment of chronic conditions. In certain embodiments, the compounds of the invention are administered for time periods exceeding two weeks, three weeks, one month, two months, three months, four months, five months, six months, one year, two years, three years, four years, or five years, ten years, or fifteen years; or for example, any time period range in days, months or years in which the low end of the range is any time period between 14 days and 15 years and the upper end of the range is between 15 days and 20 years (e.g., 4 weeks and 15 years, 6 months and 20 years). In some cases, it may be advantageous for the compounds of the invention to be administered for the remainder of the patient's life. In preferred embodiments, the patient is monitored to check the progression of the disease or disorder, and the dose is adjusted accordingly. In preferred embodiments, treatment according to the invention is effective for at least two weeks, three weeks, one month, two months, three months, four months, five months, six months, one year, two years, three years, four years, er five years, ten years, fifteen years, twenty years, or for the remainder of the subject's life.

In one embodiment, the invention provides a method of monitoring treatment progress. The method includes the step of determining a level of EAAT (e.g., EAAT2) expression in a subject suffering from or susceptible to a disorder or symptoms thereof associated with neurodegeneration, in which the subject has been administered a therapeutic amount of an amount of a β-lactam compound sufficient to treat the disease or symptoms thereof associated with neurodegeneration. The level of EAAT expression determined in the method can be compared to known levels of EAAT expression in either healthy normal controls or in other afflicted patients to establish the subject's disease status. In preferred embodiments, a second level of EAAT expression in the subject is determined at a time point later than the determination of the first level, and the two levels are compared to monitor the course of disease or the efficacy of the therapy. In certain preferred embodiments, a pre-treatment level of EAAT expression in the subject is determined prior to beginning treatment according to this invention; this pre-treatment level of EAAT expression can then be compared to the level of EAAT expression in the subject after the treatment commences, to determine the efficacy of the treatment.

IV. Kits

The invention also provides kits for treatment or prevention of a disease or disorder (or symptoms) thereof associated with neurodegeneration. In one embodiment, the kit includes an effective neuroprotective amount of a β-lactam compound in unit dosage form, together with instructions for administering the β-lactam compound to a subject suffering from or susceptible to a disease or disorder or symptoms thereof associated with neurodegeneration, wherein the effective neuroprotective amount of a beta-lactam compound is less than 250 mg of the compound. In preferred embodiments, the kit comprises a sterile container which contains the beta-lactam compound; such containers can be boxes, ampules, bottles, vials, tubes, bags, pouches, blister-packs, or other suitable container form known in the art. Such containers can be made of plastic, glass, laminated paper, metal foil, or other materials suitable for holding medicaments. The instructions will generally include information about the use of the beta-lactam compound for treatment of a disease or disorder or symptoms thereof associated with neurodegeneration; in preferred embodiments, the instructions include at least one of the following: description of the β-lactam compound; dosage schedule and administration for treatment of a disease or disorder or symptoms thereof associated with neurodegeneration; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The invention will be further described in the following examples. It should be understood that these examples are for illustrative purposes only and are not to be construed as limiting this invention in any manner.

### EXAMPLES

### Example 1

### In vitro Analysis of EAAT2 Protein Expression

Screening Assay for EAAT2 Protein overexpression. Spinal cord organotypic cultures and astroglial cultures are used to screen for drugs capable of stimulating EAAT2 synthesis and function. Organotypic cultures offer the advantage in that they maintain the normal architecture of neuron-astroglial interactions in vitro and are derived from post natal tissue; thus may better reflect astroglial responses in vivo (rather than embryonic cells). Thus a drug that acts either on an astrocyt—or induces neurons to secrete factors that alert astrocytes—better reflects the “natural” condition of delivering a drug to a whole animal.

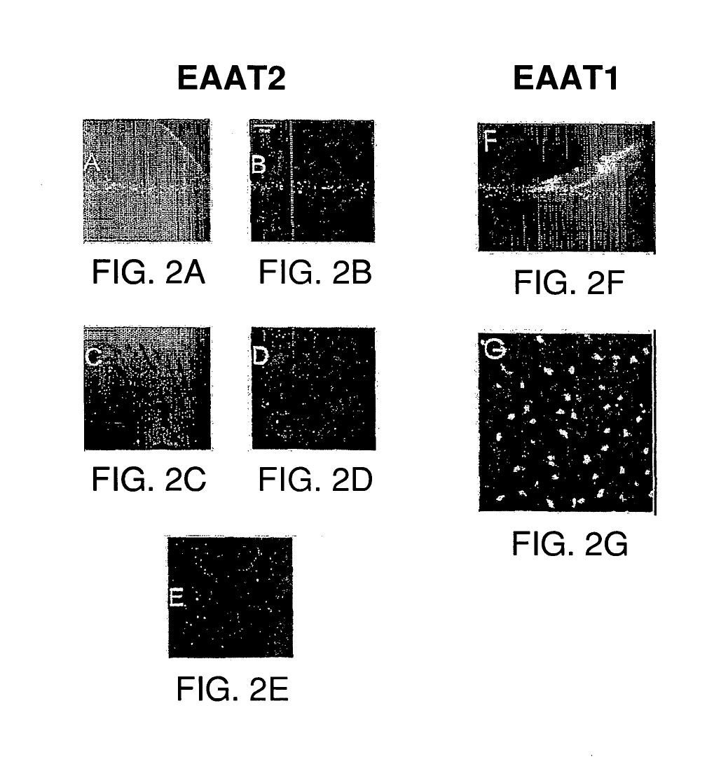

Bioassay Method (see FIG. 2 for Assay Description Summary)

Organotypic Spinal Cord. Spinal cord organotypic cultures have been described by us in detail in the past. Rothstein J D et al., N. Engl. J. Med. 1992; 326:1464-1468. Briefly, 300 um sections of rat lumbar spinal cord, from postnatal day 8-10 rat pups, are placed on Millipore Millicell CM semipermeable membranes. Each well contains 5 slices (FIG. 2A). Fifty-100 cultures can be prepared weekly. Each drug (10-100 μM) was added for 3-7 days, along with cell culture medium/serum. Cultures were harvested and 5-50 μg of tissue was applied to slot blot apparatus for detection of EAAT2 by standard Western blotting/chemiluminescence methods described in the past. Kuncl R W et al., Motor neuron diseases. In: Asbury A K et al., editors. Diseases of the Nervous System. 2 ed. Philadelphia: W. B. Saunders, 1992:1179-1208; Rothstein J D et al., Neuron 1994; 13:713-725. All antipeptide antibodies were affinity purified and highly specific for transporter subtypes. A typical slot-blot analysis, is shown in FIG. 2B,C. By this method, increases greater than 50% of expressed protein can be detected. For each antibody slot blot, the homogenates used are expected to be within the linear range for antibody detection, based on prior standard curves.

Screening Library-Assay Design. The library of compounds for these first studies was the NINDS Custom Collection from Microsource Discovery. The library is composed of 1040 compounds in 96 well plates, that also included positive control for transporter synthesis (dibutyryl cyclic AMP [dbcAMP], GDNF). These compounds (dbcAMP, GDNF, GPI-1046) induced a large increase in EAAT2 expression after 3 days in culture. The library is a unique collection of known bioactive compounds that permit the simultaneous evaluation of hundreds of marketed drugs and biochemical standards. Each compound was studied at a final concentration of 10-100 μM. All assays were performed in duplicate. Every blot included control—untreated tissue and a positive control, such as those identified above. A typical slot blot is shown in FIG. 2C.

Data Analysis. All blots were analyzed by laser densitometry (BioRad Image Quant) and the duplicate points were averaged. The complete result dataset from the 1040 compounds is shown in FIG. 2D. Each blot included a positive control standard (e.g. dbcAMP) and a negative control standard (e.g. serum, DMSO). Data was kept in Excel Spreadsheets, using a numerical/text coding system. All positive drugs (positive defined as at least a 50% increased in protein expression) were re-evaluated.

RESULTS: Screened Drugs Can Increase EAAT2 In vitro. After screening 1040 compounds, we were able to identify more than 10 related compounds capable of increasing EAAT2 protein levels by 3.5 to 7 fold (see FIG. 2E). In total, we identified 80 compounds capable of increasing EAAT2 by 2 fold or more in the first screen. Of that list, β-lactam antibiotics were overly represented and were the most common structural motif observed in all compounds—15 different beta lactam antibiotics, including penicillin and derivatives, were active. As shown in FIG. 2E, these β-lactams were all capable of increased EAAT2 protein expression. A follow-up dose response analysis (FIG. 2F) revealed and EC₅₀ for protein expression for ceftriaxone of 3.5 μM.

### Example 2

### EAAT2 Promoter Reporter Activation

Generation of COS7 and Human Astroglial Promoter reporter cell lines.

EAAT2 promoter (E2P) isolation and Reporter Generation. A 2.7 kb EAAT2 promoter fragment was obtained by cutting the PAC clone RP4-683L5 with Kpn1 and Nco1. Previous studies document that sequence, 5′ of EAAT2 coding region, has promoter motifs and can be activated in vitro. The promoter was cloned into the pGL3-basic luciferase reporter vector (Promega) (referred to as pE2P-GL3) or pEGFP-1 plasmid (Clontech) (named pE2P-eGFP). E2P was also cloned into a pLck-eGFP plasmid (a myristoylated version of eGFP that targets the eGFP protein to the membrane). Finally, E2P was also cloned into a pE2P-Luciferase-IRES-Lck-eGFP plasmid, which has the fragment E2P-Luciferase from pE2P-GL3, followed by an IRES (internal ribosomal entry site), followed by Lck-eGFP (named pELILE). In this last construct, E2P drives the expression of both Luciferase and eGFP at the same time.

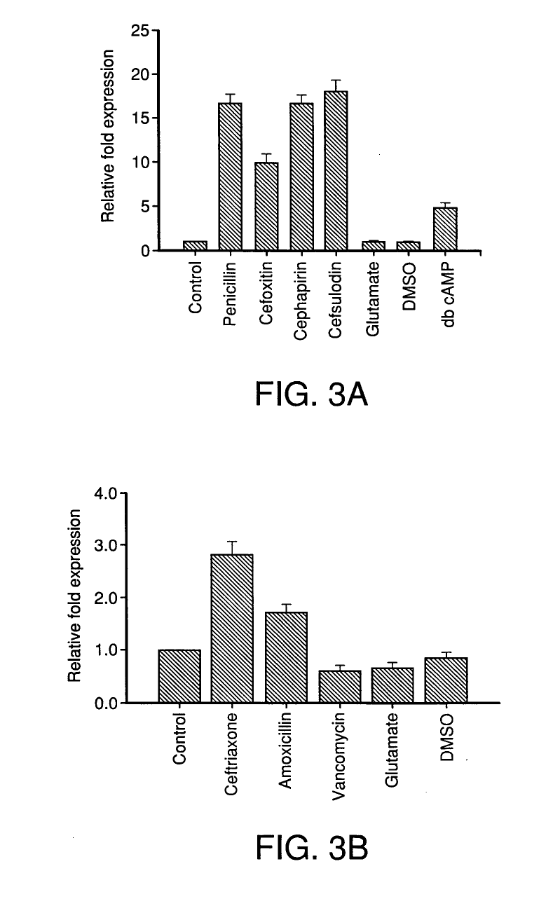

Generation of E2P-eGFP and Bac-EAAT1-eGFP Transgenic mice (FIG. 3). To provide screening cell lines for the assays we have now successfully generated two transgenic mice that express the EAAT2 promoter fragment (E2P) or the full length EAAT1 promoter (Bac-EAAT1). As shown in FIG. 3 we have generated E2P transgenic mice that demonstrate widespread expression of the EAAT2 promoter reporter in the CNS. Similar we generated Bac-EAAt1 mice and expressing cells. Recently the Heintz group also generated an EAAT1 Bac-reporter based mouse based on a similar Bac construct used in our own mice.

Screening assays. Lipofectamine 2000 reagent was used to transfect Cos-7 and HEK-293 cells. Human cortical astroglial cell were obtained from our colleague, Dr. Avi Nath. The EpE2P-eGFP, pE2P-Lck-eGFP, and pELILE contain a Neomycin resistance gene that permitted establishment of stable cell lines. For the human astroglial cells, SV40 was used to immortalize the cell. Stably transfected cells were seeded on 24-well plates, incubated with 10 uM compound solution for 48 hours and the fluorescence intensity was recorded with an automated reader (SpectraGeminiXS).

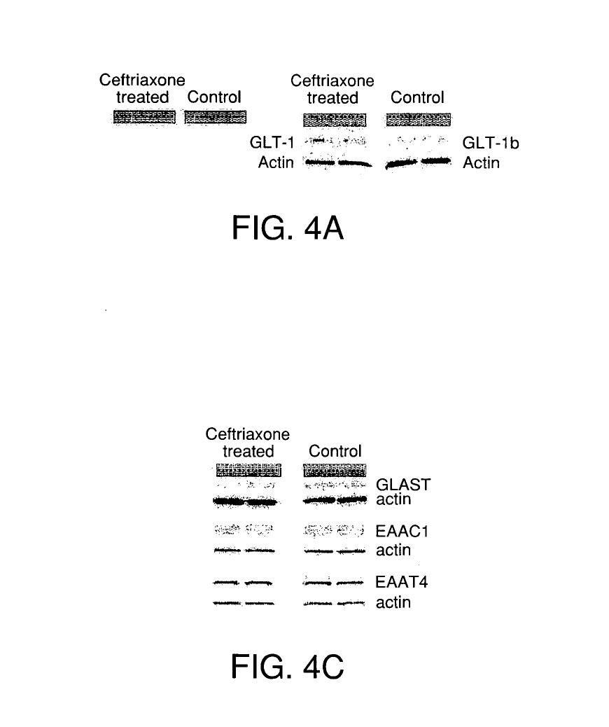

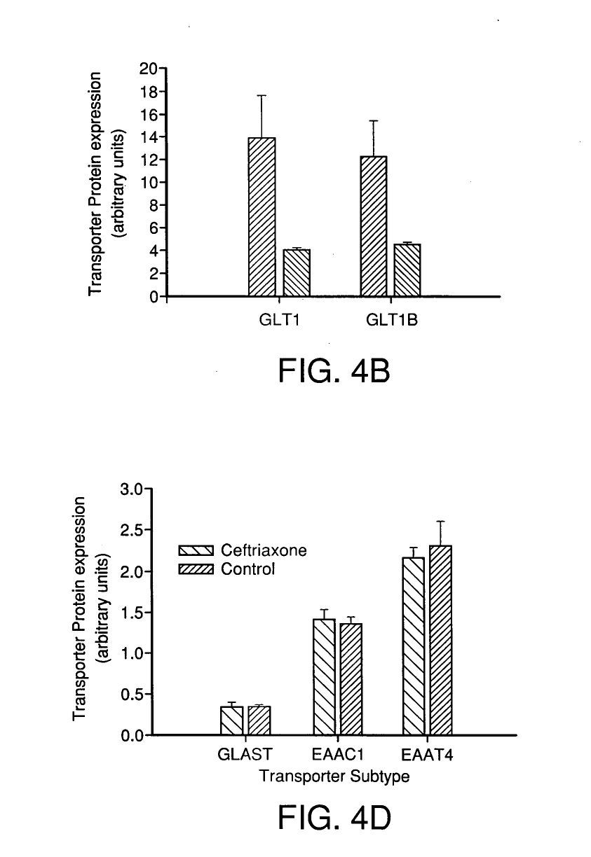

RESULTS: Identification of EAAT2 Promoter activating compounds (FIG. 4). From the original NINDS screen, we identified numerous β lactam compounds capable of potently activating EAAT2 promoter. As shown in FIG. 4, most β-lactams were able to increase EAAT promoter—far more than the known positive control, dibutyrl cyclic AMP. All compounds were active at a pharmacologically relevant concentration of 1-10 uM—a concentration range that these compounds can be found in the CNS after standard anti-bacterial therapy (e.g. ceftriaxone).

### Example 3

### In vivo Activation of EAAT Expression/Function

In vivo activation of protein expression/function can be assessed as delineated in the example below using ceftriaxone as the test compound.

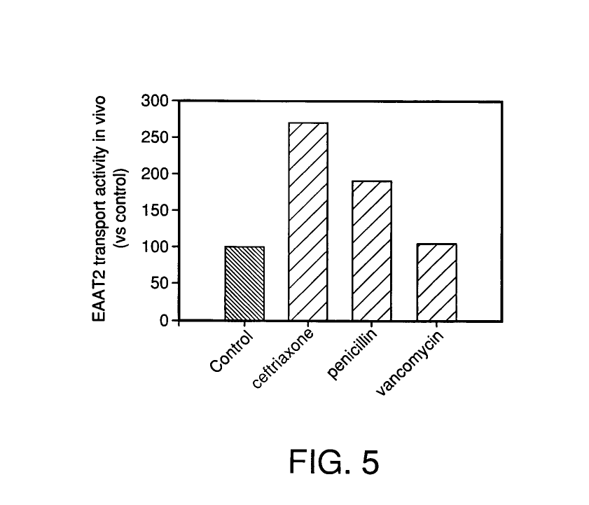

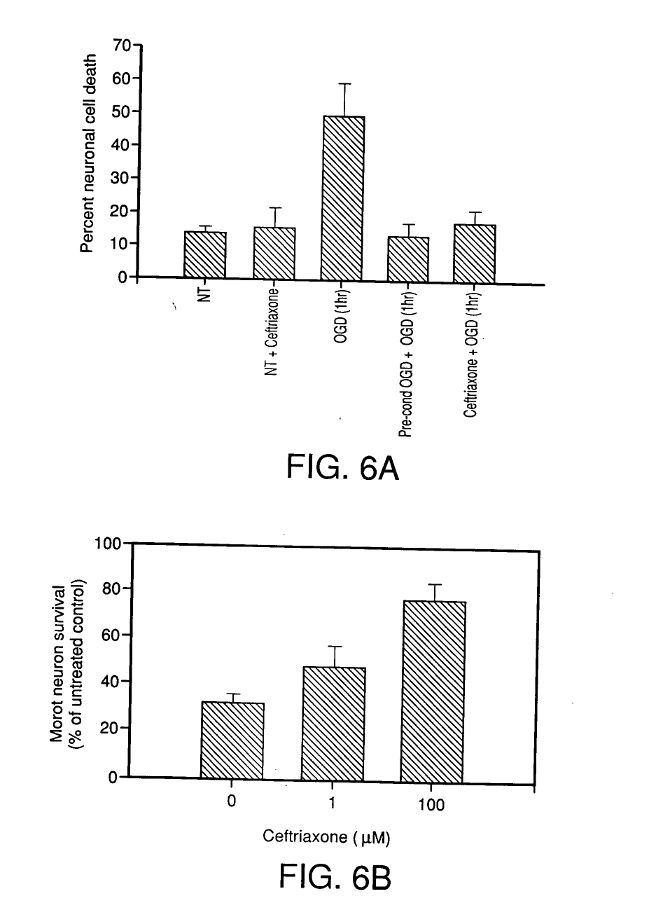

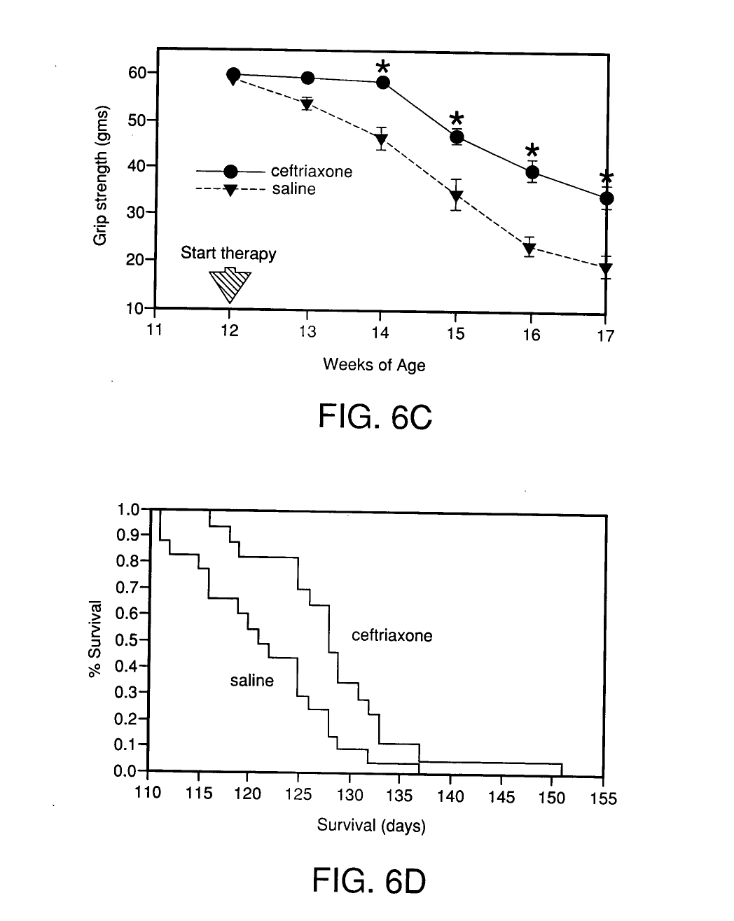

Ceftriaxone Increases Brain GLT1 level (FIG. 5). To determine if a drug identified as described herein could actually induce EAAT expression in vivo, we administered ceftriaxone to rats (n=5) (and mice, n=3) daily. Ceftriaxone was administered at a dose known to lead to CNS levels, 200 mg/kg ip. After 5 days of chronic daily administration animals were sacrificed and brain tissue harvested. As shown in FIG. 5 A,B, ceftriaxone therapy lead to 3 fold increase in brain GLT1 levels, as well as its normal splice product, GLT1b. This increase is comparable to the promoter activation results seen in vitro (FIG. 4). Western blots for the astroglial glutamate transporter GLAST as well as the two neuronal glutamate transporters, EAAC1 and EAAT4, showed no alteration in transporter expression after ceftriaxone therapy (FIG. 5C,D). Similarly, the constitutive protein, actin, was unchanged by ceftriaxone administration (FIG. 5A,C).

### Example 4

### Neuroprotection of Compounds

To evaluate the potential neuroprotection afforded by increased expression of EAAT2 by promoter activating drugs, we have conducted several in vitro and in vivo experiments—where glutamate toxicity contributes to neuronal death. Neuroprotection can be assessed as delineated in the example below using β-lactam antibiotics as the test compound.

In Vitro Model of Ischemia—Oxygen glucose deprivation (FIG. 7A) The in vitro model of oxygen glucose deprivation (OGD) is a well known and well accepted model of acute neural injury. In our in vitro model of ischemia, one hour of oxygen glucose deprivation (OGD) is lethal to cultured neurons, with toxicity known to involve excess glutamate. However, when these cultures are preconditioned 24 hours prior to the lethal condition with transient OGD (5 minutes), there is a dramatic and robust resistance of neurons to cell death. The data indicate that this neuroprotection may be due, in part, to increased expression of GLT1.

Method. Primary cortical mixed neuronal-glial cell cultures are prepared from gestation day 14-16 CD1 mice. The preparation of these cultures from mouse fetal cortex is well-described. Experiments are performed at days in vitro 13-15. In the experimental condition, cultures are subjected to oxygen glucose deprivation (OGD), an in-vitro model of ischemia. Cortical cells are either subjected to control treatment (media, modified Earle's balanced salt solution including glucose and bubbled with 5% CO₂ 95% O₂, is changed alongside treatment groups, but no OGD is performed), or 5 minutes (sublethal) of OGD (using modified Earle's balanced salt solution which is devoid of glucose and bubbled with 10% H₂, 85% N₂, and 5% to deoxygenate). Anaerobic conditions are achieved using an anaerobic chamber at 37° C. OGD is terminated by exchange of media back to oxygenated growing medium. Twenty-four hours following the above, cortical cells are subjected either to no treatment, or one hour of OGD. Neuronal survival is determined by computer-assisted cell counting after staining with the fluorescent vital dyes propidium iodide (as an indicator of neuronal death) and Hoechst 33342 (as an indicator of total number of neurons) and is presented as percent of cell death. Glial nuclei fluoresce at a lower intensity and are gated out. Drugs are added (Ceftriaxone 1 μM) 24 hours prior to the first experimental condition, and thus have been in the culture medium 48 hours prior to onset of 1 hour OGD. Following 1 hour OGD, cells are returned to growing medium without drugs.

Ceftriaxone Neuroprotection. Baseline neuronal death in the cultures is 14%, as shown in the no treatment column (NT) of FIG. 7A. Data are presented as average neuronal death in separate wells of one experiment. 1 μM Ceftriaxone, when added for 48 hours in these cultures, does not increase the baseline cell death (NT+Ceftriaxone). When cultures are subjected to 1 hour OGD, neuronal cell death, as expected, increases dramatically to approximately 50%. When cultures are preconditioned with 5 minutes of OGD 24 hours prior to 1 hour OGD, percent cell death is comparable to no treatment condition, indicating ischemic tolerance of neurons in this condition. This is the well known phenomenon of ischemic tolerance. Importantly, 1 μM Ceftriaxone, when added 48 hours prior to 1 hour OGD, also protects neurons from cell death, reducing the percentage of neuronal cell death from 50% to 20% (similar to ischemic tolerance neuroprotection). Acute administration of ceftrixone was not protective. Comparable neuroprotection was seen with the beta-lactam antibiotic cefuroxime. Thus, beta-lactam pretreatment appears to prevent neuronal death in ischemic tolerance.

In vitro model of chronic motor neurodegeneration (FIG. 7B). A model of chronic neurodegeneration was used, based on the blockade of glutamate transport in spinal cord organotypic cultures, with the non specific inhibitor threo-hytdroyxaspartate (THA) or TBOA. Chronic incubation of cultures with THA (or TBOA) leads to chronic increase in extra cellular glutamate and subsequent slow death of motor neurons (over 4 weeks). The organotypic spinal cord culture model was developed to study aspects of glutamate-mediated toxicity (and therapy). It has been useful in pre-clinical drug identification (including—riluzole—the only FDA approved drug for ALS, and more recently—celecoxib). Increased expression of glutamate transporter GLT1, by genetic over expression (e.g. transfection or transgenic over expression), in this system, can prevent motor neuron death (not shown) and neuronal death in transgenic animals. Guo H., et al. Hum Mol. Genet. 2003; 12:2519-2532.

To determine if drug induced GLT1 promoter activation, and the subsequent over expression of GLT1 protein could be neuroprotective, we used the organotypic spinal cord paradigm. Organotypic spinal cord cultures were prepared from lumbar spinal cords of 8-day-old rat pups, as described previously. Rothstein J D, et al. Proc Natl Acad Sci USA. 1993; 90:6591-6595. Ceftriaxone was added with media changes. No drugs were added for the first 7 days following culture preparation. THA was then added to experimental cultures at a concentration of 100 μM, which produces death of motor neurons within 3 to 4 weeks. Various concentrations of ceftriaxone were added as indicated, to achieve final concentrations from 0 to 100 μM. Experiments were always performed with control spinal cord cultures (ie—no drugs added), THA alone, ceftriaxone alone, and ceftriaxone+THA. Experiments at each concentration of ceftriaxone were repeated 3-5 times. The medium, with THA and ceftriaxone at the indicated concentrations, was changed twice a week. After 4 weeks, cultures were fixed, and immuostained for neurofilament (SMI-32, Sternberger) to quantify large ventral horn motor neurons (a well established method to follow motor neuron survival in this system).

Neuroprotection by ceftriaxone. As shown in FIG. 7B, ceftriaxone treatment prevented motor neurons loss in a dose dependent manner. As shown in the previous Examples, this concentration of ceftriaxone increases GLT1 protein and function by at least 3 fold. Importantly, the concentrations used in these studies are within the range attainable with oral/parenteral administration of ceftriaxone (14 grams/day). Notably, neuroprotection cannot be seen in cultures prepared from GLT-1 null mice (not shown). Similar neuroprotective effects were seen with penicillin, but not with vancomycin, which did not alter GLT1 levels.

In Vivo Neuroprotection—Effect of ceftriaxone on onset and progression of motor neuron disease in the G93A SOD1 Mouse. (FIG. 7C,D)

To determine if ceftriaxone could alter neurodegeneration in a disease model that involves altered expression of glutamate transporters we treated G93A SOD1 mice with ceftriaxone. Numerous studies have documented a contributory role for excess glutamate in this mouse model—and role for modulating glutamate receptors or transporters in neuroprotective strategies. Guo H., et al. Hum Mol. Genet. 2003; 12:2519-2532. Modest over expression—by a transgenic approach—can alter disease onset and/or survival. Furthermore, recent studies suggest that late administration of drugs, e.g. at time of disease onset, may be more therapeutically relevant.

Treatment paradigm. G93A SOD1 mice [(B6.Cg-Tg (SOD1-G93A)1Gur/J, high expresser] were treated with ceftriaxone (200 mg/kg ip) starting at approximately 12 weeks of age. Drug treated animals (n=20) and saline injected controls (n=20) were monitored daily for survival and weekly for grip strength (Columbus Instruments) and for body weight, as described previously.

Ceftriaxone delays loss of Grip Strength and Increases survival. As shown in FIG. 7C, ceftriaxone treatment significantly delayed loss of muscle strength. This effect was observed within 7 days after treatment, and persisted for 4 weeks; by 18 weeks of age the strength preservation was lost. In a similar manner, the drug also increased over all survival of the mice by about 7-10 days (FIG. 7D). Although this effect is relatively small, the drug was given at the time of disease onset, and thus, even a small effect may have clinical significance. When the same dose of drug was administered somewhat earlier, at 42 days of age, survival was also increased, although not significantly better than late delivery at 90 days of age. The lack of greater efficacy when given earlier would be consistent with the observation that the loss of GLT1 expression does not begin to occur until around 90 days in this model.

To determine if ceftriaxone altered cellular degeneration in vivo, around the time of clinical onset, G93A mice were treated with ceftriaxone starting at 70 days of age. Two weeks of drug therapy lead to a significant prevention of motor neuron loss, and reduction of the hypercellular-associated gliosis and inflammation compared to saline treated control G93A mice. GLT1 expression decreases around the onset of clinical disease, yet ceftriaxone administration was able to increase endogenous GLT1 expression significantly in spinal cords from the chronically treated mice. The neuroprotection seen in this study is not likely to be due to the normal antibiotic properties of the drug, since ALS mice are not septic and do not typically have lung infections at 12-16 weeks of age, when prominent muscle strength effects were seen.

Treatment of ALS with Ceftriaxone

Patients suffering from amyotrophic lateral sclerosis (ALS) are treated with ceftriaxone (10 mg/kg intravenous once per day). Patients receive periodic physical and neurological exams, including tests such as electromyography, manual muscle testing, respiratory function measurement, and nerve conduction velocity tests, to evaluate the course of the disease. Additionally, EAAT2 levels and/or blood or CSF levels of ceftriaxone are periodically assessed. The dosage of ceftriaxone is adjusted (increased or decreased) as appropriate based on the test results and clinical observation.

PCT Patent Applications PCT/US03/04414 and PCT/US04/005698 are expressly incorporated by reference herein and provide further screening and analytical protocol information.

All references cited herein, whether in print, electronic, computer readable storage media or other form, are expressly incorporated by reference in their entirety, including but not limited to, abstracts, articles, journals, publications, texts, treatises, technical data sheets, internet web sites, databases, patents, patent applications, and patent publications.

A number of embodiments of the invention have been described. Nevertheless, it will be understood that various modifications may be made without departing from the spirit and scope of the invention. Accordingly, other embodiments are within the scope of the following claims.

1. A method of treating a subject suffering from or susceptible to a disease or disorder associated with neurodegeneration, the method comprising the step of administering to the subject a therapeutic amount of a beta-lactam compound which is sufficient to treat the disease or disorder or symptoms thereof associated with neurodegeneration under conditions such that the disease or disorder associated with neurodegeneration is treated, but which does not result in substantial clinically effective antibiotic activity.

2. The method of claim 1, wherein the subject is a human.

3. The method of claim 1, wherein the subject is a subject identified as being in need of such treatment.

4. The method of claim 1, wherein the subject is not suffering from a bacterial infection.

5. The method of claim 1, wherein the step of administering the beta-lactam compound comprises administering the beta-lactam compound for a period of greater than 2 weeks.

6. The method of claim 1, wherein the step of administering the beta-lactam compound comprises administering the beta-lactam compound for a period of at least about 6 months.

7. The method of claim 1, wherein the step of administering the beta-lactam compound comprises administering the beta-lactam compound in a dosage of less than about 500 mg/day.

8. (canceled)

9. The method of claim 1, wherein the average plasma concentration of the beta-lactam compound in the subject does not exceed about 10 micrograms per milliliter.

10. The method of claim 1, wherein the beta-lactam compound is a cephalosporin.

11. The method of claim 10, wherein the cephalosporin is ceftriaxone or a salt thereof.

12. The method of claim 10, wherein the beta-lactam compound is ceftriaxone sodium.

13. The method of claim 11, wherein the beta-lactam compound is ceftriaxone disodium salt, sesquaterhydrate.

14. The method of claim 1, wherein EAAT2 protein expression is increased in vivo.

15. The method of claim 14, wherein EAAT2 production is increased by 200% or more relative to non-regulated production.

16. The method of claim 1, wherein the disease or disorder associated with neurodegeneration is selected from the group consisting Parkinson's disease, Huntington's disease, Alzheimer's disease, multiple sclerosis, amyotrophic lateral sclerosis, acute neurological diseases, epilepsy, spinal cord injury, brain trauma, glaucoma, peripheral neuropathy, and psychiatric disorders.

17. The method of claim 1, wherein the step of administering comprises administering the compound intravenously or intramuscularly.

18. A kit comprising an effective neuroprotective amount of a beta-lactam compound in unit dosage form, together with instructions for administering the beta lactam compound to a subject suffering from or susceptible to a disease or disorder or symptoms thereof associated with neurodegeneration, wherein the effective neuroprotective amount of a beta-lactam compound is an amount which does not result in substantial clinically effective antibiotic activity.

19. The kit of claim 18, wherein the beta-lactam compound is a cephalosporin.

20. The kit of claim 18, wherein the beta-lactam compound is ceftriaxone, or a salt thereof.

21. The method of claim 1, further comprising determining a level of EAAT expression in the subject.

22. The method of claim 21, wherein the determining of the level of EAAT expression is performed prior to administration of the beta-lactam compound to the subject.

23. The method of claim 21, wherein the determining of the level of EAAT expression is performed subsequent to administration of the beta-lactam compound to the subject.

24. The method of claim 21, wherein the determining of the level of EAAT expression is performed prior to and subsequent to administration of the beta-lactam compound to the subject.

25. The method of claim 24, wherein the levels of EAAT expression performed prior to and subsequent to administration of the beta-lactam compound to the subject are compared.

26. The method of claim 25, wherein the comparison of EAAT levels is reported by a clinic, laboratory, or hospital agent to a health care professional.

27. The method of claim 24, wherein when the level of EAAT expression performed prior to administration of the beta-lactam compound to the subject is lower than the level of EAAT expression performed subsequent to administration of the beta-lactam compound to the subject, then the amount of compound administered to the subject is an effective amount.

28. The method of claim 7, wherein the step of administering the beta-lactam compound comprises administering the beta-lactam compound in a dosage of less than about 250 mg/day.

29. The method of claim 28, wherein the step of administering the beta-lactam compound comprises administering the beta-lactam compound in a dosage of less than about 100 mg/day.

30. The method of claim 29, wherein the step of administering the beta-lactam compound comprises administering the beta-lactam compound in a dosage of less than about 50 mg/day.

31. The method according to claim 1 wherein the beta-lactam compound is administered in combination with at least one other pharmacological agent.

32. The method according to claim 31, wherein the at least one other pharmacological agent is a non-steroidal anti-inflammatory compound; riluzole; levodopa; a dopa agonist; an acetylcholinesterase inhibitor; an NMDA receptor blocker; gabapentin; amytriptyline; or an interferon.

33. The method according to claim 32, wherein the non-steroidal anti-inflammatory compound is aspirin, naproxen, sulindac, diclofenac, ibuprofen, celecoxib or valdecoxib.

34. The method according to claim 32, wherein the NMDA receptor blocker is memantine.

35. The method of claim 16, wherein the psychiatric disorder is depression or bipolar disorder.

36. A kit according to claim 18, wherein the amount of beta-lactam compound in unit dosage form is less than 250 mg.
